SEQUENCE LISTING (38 sequences)

SEQ ID NO: 1 - DNA, 21 bp, Artificial Sequence
  Synthetic polynucleotide
  misc_RNA (1)..(19): siRNA sense strand ribonucleotides
  misc_feature (20)..(21): T = deoxythymidine  siRNA sense strand
ccgagccacaucgcucagatt

SEQ ID NO: 2 - DNA, 21 bp, Artificial Sequence
  Synthetic polynucleotide
  misc_RNA (1)..(19): Polyribonucleotide  siRNA antisense strand
  misc_feature (20)..(21): T = deoxythymidine  siRNA antisense strand
ucugagcgauguggcucggtt

SEQ ID NO: 3 - DNA, 24 bp, Artificial Sequence
  Synthetic polynucleotide
  misc_RNA (1)..(19): Polyribonucleotide  siRNA sense strand
  misc_feature (20)..(24): T = deoxythymidine  siRNA sense strand
ccgagccacaucgcucagattttt

SEQ ID NO: 4 - DNA, 27 bp, Artificial Sequence
  Synthetic polynucleotide
  misc_RNA (1)..(19): Polyribonucleotide  siRNA sense strand
  misc_feature (20)..(27): T = deoxythymidine  siRNA sense strand
ccgagccacaucgcucagatttttttt

SEQ ID NO: 5 - DNA, 24 bp, Artificial Sequence
  Synthetic polynucleotide
  misc_RNA (1)..(19): Polyribonucleotide  siRNA antisense strand
  misc_feature (20)..(24): T = deoxythymidine  siRNA antisense strand
ucugagcgauguggcucggttttt

SEQ ID NO: 6 - DNA, 24 bp, Artificial Sequence
  Synthetic polynucleotide
  misc_RNA (1)..(19): Polyribonucleotide  siRNA sense strand
  misc_feature (20)..(24): A = deoxyadenosine  siRNA sense strand
ccgagccacaucgcucagaaaaaa

SEQ ID NO: 7 - DNA, 24 bp, Artificial Sequence
  Synthetic polynucleotide
  misc_RNA (1)..(19): Polyribonucleotide  siRNA sense strand
  misc_feature (20)..(24): G = deoxyguanosine  siRNA sense strand
ccgagccacaucgcucagaggggg

SEQ ID NO: 8 - DNA, 24 bp, Artificial Sequence
  Synthetic polynucleotide
  misc_RNA (1)..(19): Polyribonucleotide  siRNA sense strand
  misc_feature (20)..(24): C = deoxycytosine  siRNA sense strand
ccgagccacaucgcucagaccccc

SEQ ID NO: 9 - RNA, 24 nt, Artificial Sequence
  Synthetic polynucleotide
ccgagccacaucgcucagaaaaaa

SEQ ID NO: 10 - RNA, 21 nt, Artificial Sequence
  Synthetic polynucleotide
ucugagcgauguggcucgguu

SEQ ID NO: 11 - RNA, 19 nt, Artificial Sequence
  Synthetic polynucleotide
ucugagcgauguggcucgg

SEQ ID NO: 12 - DNA, 24 bp, Artificial Sequence
  Synthetic polynucleotide
  misc_RNA (1)..(19): Polyribonucleotide  siRNA sense strand
  misc_binding (1)..(1): C is labelled with Cy3
  misc_feature (20)..(24): T = deoxythymidine siRNA sense strand
ccgagccacaucgcucagattttt

SEQ ID NO: 13 - DNA, 21 bp, Artificial Sequence
  Synthetic polynucleotide
  misc_RNA (1)..(19): Polyribonucleotide  siRNA sense strand
  misc_feature (20)..(21): T = deoxythymidine  siRNA sense strand
gcaagcugacccugaaguutt

SEQ ID NO: 14 - DNA, 21 bp, Artificial Sequence
  Synthetic polynucleotide
  misc_RNA (1)..(19): Polyribonucleotide  siRNA antisense strand
  misc_feature (20)..(21): T = deoxythymidine siRNA antisense strand
aacuucagggucagcuugctt

SEQ ID NO: 15 - DNA, 24 bp, Artificial Sequence
  Synthetic polynucleotide
  misc_RNA (1)..(19): Polyribonucleotide  siRNA sense strand
  misc_RNA (1)..(19): Polyribonucleotide  cpRNA sense strand
  misc_feature (20)..(24): T = deoxythymidine  siRNA sense strand
gcaagcugacccugaaguuttttt

SEQ ID NO: 16 - DNA, 21 bp, Artificial Sequence
  Synthetic polynucleotide
  misc_RNA (1)..(19): Polyribonucleotide  siRNA sense strand
  misc_feature (20)..(21): T = deoxythymidine  siRNA sense strand
guagcugauauugauggactt

SEQ ID NO: 17 - DNA, 21 bp, Artificial Sequence
  Synthetic polynucleotide
  misc_RNA (1)..(19): Polyribonucleotide  siRNA antisense strand
  misc_feature (20)..(21): T = deoxythymidine  siRNA antisense strand
guccaucaauaucagcuactt

SEQ ID NO: 18 - DNA, 24 bp, Artificial Sequence
  Synthetic polynucleotide
  misc_RNA (1)..(19): Polyribonucleotide  siRNA sense strand
  misc_RNA (1)..(19): Polyribonucleotide  cpRNA sense strand
  misc_feature (20)..(24): T = deoxythymidine  siRNA sense strand
guagcugauauugauggacttttt

SEQ ID NO: 19 - DNA, 25 bp, Artificial Sequence
  Synthetic polynucleotide
  misc_RNA (1)..(19): polyribonucleotide  cpRNA sense strand
  misc_feature (20)..(24): T = deoxythymidine  cpRNA sense strand
  misc_RNA (25)..(25): ribonucleotide  cpRNA sense strand
ccgagccacaucgcucagatttttu

SEQ ID NO: 20 - RNA, 22 nt, Artificial Sequence
  miR-34a sense sequence
caaucagcaaguauacugcccu

SEQ ID NO: 21 - RNA, 22 nt, Artificial Sequence
  miR-34a antisense sequence
uggcagugucuuagcugguugu

SEQ ID NO: 22 - DNA, 27 bp, Artificial Sequence
  miR-34a-CP sense sequence
  misc_RNA (1)..(22): polyribonucleotide
  misc_feature (23)..(27): A = deoxyadenosine
caaucagcaaguauacugcccuaaaaa

SEQ ID NO: 23 - RNA, 22 nt, Artificial Sequence
  miR-34a-CP antisense sequence
uggcagugucuuagcugguugu

SEQ ID NO: 24 - DNA, 26 bp, Artificial Sequence
  siR-34a-CP sense sequence
  misc_RNA (1)..(21): polyribonucleotide
  misc_feature (22)..(26): A = deoxyadenosine
aaucagcuaaguauacugccaaaaaa

SEQ ID NO: 25 - RNA, 23 nt, Artificial Sequence
  siR-34a-CP antisense sequence
uggcaguguacuuagcugguugu

SEQ ID NO: 26 - DNA, 24 bp, Artificial Sequence
  GAPDH siRNA sense sequence
  misc_RNA (1)..(19): polyribonucleotide
  misc_feature (20)..(24): t = deoxythymidine
ccgagccacaucgcucagattttt

SEQ ID NO: 27 - DNA, 21 bp, Artificial Sequence
  GAPDH siRNA antisense sequence
  misc_RNA (1)..(19): polyribonucleotide
  misc_feature (20)..(21): t = deoxythymidine
ucugagcgauguggcucggtt

SEQ ID NO: 28 - DNA, 24 bp, Artificial Sequence
  Nicked GAPDH CP-siRNA sense strand
  misc_RNA (1)..(19): polyribonucleotide
  misc_feature (20)..(24): t = deoxythymidine
ccgagccacaucgcucagattttt

SEQ ID NO: 29 - RNA, 10 nt, Artificial Sequence
  Nicked GAPDH CP-siRNA antisense sequence a
ucugagcgau

SEQ ID NO: 30 - RNA, 9 nt, Artificial Sequence
  Nicked GAPDH CP-siRNA antisense sequence b
guggcucgg

SEQ ID NO: 31 - RNA, 24 nt, Artificial Sequence
  CP-siRNA-5FU5 sense sequence
  misc_feature (20)..(24): u = 5-Fluoro Uracil
ggaagcugcagaaagauacuuuuu

SEQ ID NO: 32 - DNA, 21 bp, Artificial Sequence
  CP-siRNA-5FU5 antisense sequence
  misc_RNA (1)..(19): polyribonucleotide
  misc_feature (20)..(21): t = deoxythymidine
guaucuuucugcagcuucctt

SEQ ID NO: 33 - RNA, 21 nt, Artificial Sequence
  siRNA-5FU2 sense sequence
  misc_feature (20)..(21): u = 5-Fluoro Uracil
ggaagcugcagaaagauacuu

SEQ ID NO: 34 - DNA, 21 bp, Artificial Sequence
  siRNA-5FU2 antisense sequence
  misc_RNA (1)..(19): polyribonucleotide
  misc_feature (20)..(21): t = deoxythymidine
guaucuuucugcagcuucctt

SEQ ID NO: 35 - DNA, 25 bp, Artificial Sequence
  Mod3a 5p strand
  misc_RNA (1)..(22): polyribonucleotide
  misc_feature (23)..(25): t = deoxythymidine
uggcagugucuuagcugguuguttt

SEQ ID NO: 36 - DNA, 27 bp, Artificial Sequence
  Mod3b 5p strand
  misc_RNA (1)..(22): polyribonucleotide
  misc_feature (23)..(27): t = deoxythymidine
uggcagugucuuagcugguuguttttt

SEQ ID NO: 37 - DNA, 25 bp, Artificial Sequence
  Mod5a 3p strand
  misc_RNA (1)..(22): polyribonucleotide
  misc_feature (23)..(25): t = deoxythymidine
caaucagcaaguauacugcccuttt

SEQ ID NO: 38 - DNA, 27 bp, Artificial Sequence
  Mod5b 3p strand
  misc_RNA (1)..(22): polyribonucleotide
  misc_feature (23)..(27): t = deoxythymidine
caaucagcaaguauacugcccuttttt

(19) United States
(12) Patent Application Publication
(10) Pub. No.: US 2018/0265867 A1
(43) Pub. Date: Sep. 20, 2018

(54) NUCLEIC ACID MOLECULES WITH ENHANCED ACTIVITY

(71) Applicants: Oommen P. Oommen, Uppsala (SE); Oommen P. Varghese, Uppsala (SE)

(72) Inventors: Oommen P. Oommen, Uppsala (SE); Oommen P. Varghese, Uppsala (SE)

(21) Appl. No.: 15/761,753

(22) PCT Filed: Sep. 21, 2016
(86) PCT No.: PCT/EP2016/072466

§ 371 Date: Mar. 20, 2018

(30) Foreign Application Priority Data
Sep. 21, 2015 (GB) 1516685.3

(51) Int. Cl. C12N 15/113

(52) U.S. Cl. CPC C12N 15/113; C12N 2310/14; C12N 2310/533; C12N 2310/20; C12N 2310/141

(57) ABSTRACT

Double-stranded nucleic acid molecules (100, 200, 300, 400, 500 and 600) are provided comprising a sense strand (110, 210, 310, 410, 510 and 610) and an antisense strand (120, 220, 320, 420, 520 and 620). The sense strand has a 3′-overhang (115, 215, 315, 415, 515 and 615) of from four to eight nucleotides. The double-stranded nucleic acid molecules include double-stranded RNA (dsRNA), double-stranded DNA (dsDNA), double-stranded DNA-RNA (sense-antisense) hybrid and double-stranded RNA-DNA (sense-antisense) hybrid. The double-stranded nucleic acid molecules have beneficial properties in terms of unaided cellular delivery (e.g. in the absence of a cationic agent), cellular delivery and release of the antisense strand in a cell as a single-stranded molecule and the delivery of the antisense strand into the nucleus of a cell as a single-stranded molecule. Related compositions, cells, uses and methods are also provided.

### TECHNICAL FIELD

The present embodiments generally relate to double-stranded nucleic acid molecules and in particular to double-stranded nucleic acid molecules having improved characteristics in terms of cellular delivery and function.

### BACKGROUND

Since the discovery of antisense technology, synthetic nucleic acid based gene regulating technologies have expanded enormously. Of particular interest are synthetic short interfering RNA (siRNA) and micro RNA (miRNA) as potent gene silencing molecules in mammalian cells. Drugs based on these nucleic acids have emerged as promising therapeutics to treat a variety of diseases. A typical example of miRNA that has high therapeutic potential is the miRNA-34a (miR-34a) that has been previously shown to attenuate osteoporosis and bone cancer metastasis in mouse model (Krzeszinski J Y et al, 2014, Nature, 512(7515), 431-435). This miRNA is also known to inhibit prostate cancer by downregulating CD44 gene. Currently this miRNA is pursued in different clinical trial with several formulations.

Gene-regulating strategies that modulate gene function by targeting mRNA, pre-mRNA and/or DNA and include:

Small interfering RNAs (siRNAs): siRNAs are 21-23 nucleotide-long, double-stranded RNAs with an antisense active strand that is exactly complementary to a sequence anywhere in the target mRNA. siRNAs are taken up by the cytosolic RNA-induced silencing complex (RISC), which ejects one strand, leaving the antisense strand to bind to the target mRNA and mediate its sequence-specific cleavage by the Argonaute 2 protein in the RISC. Once cleaved, the target mRNA is rapidly degraded.

Anti-miRs and miRNA mimics: Oligonucleotides can be used to antagonize (in which case they are known as anti-miRs) or mimic the function of endogenous microRNAs (miRNAs). Native miRNAs are taken up by the RISC, which suppresses gene expression of RNAs containing partially complementary sequences by blocking their translation or accelerating their degradation. They suppress the expression of hundreds of transcripts, but are less efficient than siRNAs.

Single-stranded antisense oligonucleotides (ASOs) can bind to miRNAs to block their activity. These are called anti-miRs.

RNase H dependent ASOs: RNase H dependent antisense oligonucleotides (ASOs) are single-stranded, chemically modified oligonucleotides that bind to complementary sequences in target mRNAs and reduce gene expression both by RNase H mediated cleavage of the target RNA and by inhibition of translation by steric blockade of ribosomes. The most clinically advanced ASOs are ‘gapmer’ ASOs that incorporate a 5 nucleotide-long or longer central DNA stretch between chemically modified RNA flanks. The ‘gapmer’ strategy is generally employed, since RNase H recognizes only RNA-DNA duplex and not RNA-RNA duplex. Once ASOs bind target mRNA, it triggers rapid degradation of the target mRNA. The catalytic activity of antisense DNA is however not as efficient as siRNA.

Piwi-interacting RNA (piRNA): piRNAs are 26 to 31 nucleotide long single-stranded non-coding RNA molecules that are expressed in animal cells. These piRNAs form RNA-protein complexes through interactions with piwi proteins in the nucleus and facilitate epigenetic and post-transcriptional gene silencing.

Short hairpin RNA (shRNA): shRNA is generally generated from plasmid DNA and is used to deliver siRNA; miRNA or crispr RNA (crRNA) for RNA or DNA interference applications. These shRNAs could also be delivered directly for these applications using suitable delivery agents. Unlike si or miRNA that inhibits at the mRNA levels, crRNA together with Cas9 protein can be used for genome editing, thus act as a DNA interfering molecules.

Exon-skipping ASOs: Exon-skipping ASOs are single-stranded, usually chemically modified ASOs that target intron-exon junctions (splice sites) or splicing-regulatory elements. Binding to the target site inhibits splicing at this site and forces the choice of an alternative splice site. Changing splice site leads to the translation of an alternative protein isoform that can restore stability or function to a mutated gene product.

Nucleic acid based gene regulatory molecules have been used in combination with other small molecule drugs or chemotherapeutic agents with the aim of improving the therapeutic outcome of treatment of human diseases. Examples of this include the following: Schmitz and coworkers (Wu, S-Y et al, Nucleic Acid Res., 2013, 41, 4650-59) have reported 5-fluoro-2′-deoxyuridine conjugated siRNA design. This siRNA-5FU molecule was transfected to cells using Lipofectamine 2000.

US 2014/0088300 A1 describes design of siRNA molecules incorporated with five molecules of 5-fluoro-2′-deoxyuridine at 3′ sense, or 3′ antisense strand or on both 3′-sense and antisense strands. All the siRNA sequences tested were symmetrical, that is both 3′-sense and 3′-antisense strands had same number of nucleotides.

U.S. Pat. No. 5,663,321 describes the synthesis of homo-oligomeric 5-fluorouridine and 5-fluorodeoxyuridine nucleotide prodrugs which are used as a polymeric drug delivery system for the production of FdUMP, a potent inhibitor of thymidylate synthase.

All these gene-regulating technologies have several challenges that need to be addressed before they can be used for clinical applications. The main challenges being (1) cellular delivery of such therapeutic molecules at desired concentrations at the site of action; (2) selective strand recruitment and minimize undesired off-target effects; and (3) minimizing immune activation.

Among these challenges, the key hurdle is cellular delivery. It is well established that large anionic polymers such as siRNA (≈14000 Da), being a large negatively charged molecule, generally does not pass the plasma membrane, the biological barrier that limits its therapeutic potential. Thus, extensive research is being conducted to identify delivery carriers that overcome this cellular barrier without inducing significant toxicity. The stability of single stranded ASOs and anti-miRs is also a challenge as they are quickly degraded by serum enzymes.

US 2009/0208564 describes design of asymmetric siRNA molecules where the length of sense or passenger strand is reduced from conventional 21-mer to 12-17-mer. Such asymmetry reduces off-target effect and improves RNAi activity.

EP 1 407 044 (WO0244321) describes isolated dsRNA molecules capable of target-specific RNA interference, in which each RNA strand has a length from 19 to 23 nucleotides and wherein at least one strand has a 3′-overhang from 1 to 3 nucleotides. The dsRNA molecules of EP 1 407 044 require the presence of a carrier for transfection.

EP 1486 564 describes a method for the specific selection of dsRNA molecules capable of RNA interference and having improved efficiency through increased serum stability. The sequences of the single strands of the dsRNA molecule are selected such that at each end of the dsRNA molecule the last complementary nucleotide pair is G-C or at least two of the last four complementary nucleotide pairs are G-C pairs, wherein the dsRNA molecule has an overhang of 1-4 unpaired nucleotides.

US 2012/0041049 describes isolated dsRNA molecules possessing G/C rich sequences at the 5′-end of the passenger strand or at the 3′-end of the guide strand resulting in enhanced serum stability and knockdown efficiency.

The only carrier free delivery of siRNA is described in US2004/0198640, US 2009/0209626 and WO2010/033247, where nucleotides of the siRNA molecules are extensively modified with hydrophobic groups. Examples of other siRNA molecules are described in Caplan et al., PNAS, 98, 17, 9742-9747 (2001); Kim et al., Nature Biotechnology, 23, 2, 222-226 (2005); Bolcato-Bellmin et al., PNAS, 104, 41, 16050-16055 (2007); Mok et al., Nature Materials, 9, 272-278 (2010); Schmitz and Chu, Silence, 2, 1, 1-10 (2011); Zhongping et al., 2012, 23, 5, 521-532; WO03/012052; WO2005/014782; WO2005/079533; WO2006/128739; WO2010/080129; and WO 2011/072082.

A major area of research within the field of gene regulatory technology is to identify the most biocompatible transfection reagent. This has led to the development of carrier systems like cationic polymers, virus particles or hydrophobic modifications of nucleotides for cellular delivery.

### DESCRIPTION

The invention provides improved double-stranded nucleic acid molecules comprising a sense (or passenger) strand and an antisense (or target) strand. The double-stranded nucleic acid molecule can be a double-stranded RNA (dsRNA), a double-stranded DNA (dsDNA), a double-stranded DNA-RNA (sense-antisense) hybrid or a double-stranded RNA-DNA (sense-antisense) hybrid. The double-stranded nucleic acid molecule may be configured to promote unaided cellular delivery (e.g. in the absence of a cationic agent). The double-stranded nucleic acid molecule may further be configured to promote cellular delivery and release of the antisense strand in a cell as a single-stranded molecule. The configuration of the double-stranded nucleic acid molecule may enable the delivery of the antisense strand into the nucleus of a cell as a single-stranded molecule.

The double-stranded nucleic acid molecules of the invention may be administered and taken up by cells without the use of any other agent or reagent. The double-stranded nucleic acid molecules may be taken up by cells in the absence of a transfection agent, reagent and/or vector. The double-stranded nucleic acid molecules may be taken up by cells in the absence of an agent for facilitating their release from the endosome into the cytosol. The double-stranded nucleic acid molecules may be taken up by cells in the absence of a cationic agent. The double-stranded nucleic acid molecules may be taken up by cells with or without the use of a carrier. The double-stranded nucleic acid molecules may be taken up by cells in the absence of any hydrophobic modification of the double-stranded nucleic acid molecules. In view of these properties, the double-stranded nucleic acid molecules of the invention are also referred to as cell penetrating nucleic acids (e.g. cell penetrating RNA or cpRNA).

The invention provides a double-stranded nucleic acid molecule comprising a sense strand and an antisense strand, wherein the sense strand has a 3′-overhang of at least 3 nucleotides (and preferably from four to eight nucleotides) and the antisense strand has a 3′-overhang that is shorter than the 3′-overhang of the sense strand or the antisense strand does not have a 3′-overhang.

The invention provides a double-stranded nucleic acid molecule comprising a sense strand and an antisense strand, wherein the sense strand has a 3′-overhang of at least 3 nucleotides (and preferably from four to eight nucleotides) and the antisense strand has a 3′-overhang that is shorter than the 3′-overhang of the sense strand or the antisense strand does not have a 3′-overhang, and wherein the sense strand and/or the antisense strand is a DNA strand.

The invention provides a double-stranded nucleic acid molecule comprising a sense strand and an antisense strand, wherein the sense strand has a 3′-overhang of at least 3 nucleotides (and preferably from four to eight nucleotides) and the antisense strand has a 3′-overhang that is shorter than the 3′-overhang of the sense strand or the antisense strand does not have a 3′-overhang, and wherein the double-stranded nucleic acid molecule comprises at least two nucleotide mismatches between the sense and antisense strands.

The double-stranded nucleic acid molecule may comprise a mismatch between the sense and antisense strands of at least two contiguous nucleotides or at least three contiguous amino acids. Additionally or alternatively, the double-stranded nucleic acid molecule may comprise at least two, at least three, at least four, at least five, at least six, at least seven, at least eight, at least nine or at least ten non-contiguous nucleotide mismatches between the sense and antisense strands. Preferably, the double-stranded nucleic acid molecule comprises at least two non-contiguous nucleotide mismatches between the sense and antisense strands. Preferably the sense and antisense strands are configured to enable the unaided delivery of the double-stranded nucleic acid molecule into a cell and the release of the antisense strand as a single-stranded molecule.

The invention provides a double-stranded nucleic acid molecule comprising a sense strand and an antisense strand, wherein the sense strand has a 3′-overhang of at least three nucleotides (preferably from four to eight nucleotides) and the antisense strand has a 3′-overhang that is shorter than the 3′-overhang of the sense strand or the antisense strand does not have a 3′-overhang, and wherein the double-stranded portion of the double-stranded nucleic acid molecule comprises a nick in the sense strand.

The double-stranded portion of the double-stranded nucleic acid molecule may comprise at least two, at least three, at least four, at least five, at least six, at least seven, at least eight, at least nine or at least ten nicks in the sense strand. Preferably the sense strand is configured to enable the unaided delivery of the double-stranded nucleic acid molecule into a cell and the release of the antisense strand as a single-stranded molecule (e.g. following in-situ degradation of the sense strand).

Preferably, the configuration of the double-stranded nucleic acid molecule enables the delivery of the antisense strand into the nucleus of a cell as a single-stranded molecule.

The invention provides a double-stranded nucleic acid molecule comprising a sense strand and an antisense strand, wherein the sense strand has a 3′-overhang of at least three nucleotides (preferably from four to eight nucleotides) and the antisense strand has a 3′-overhang that is shorter than the 3′-overhang of the sense strand or the antisense strand does not have a 3′-overhang, and wherein the sense strand comprises two polynucleotides each of which is hybridized to the antisense strand. Preferably, there is a gap of one or more nucleotides between the polynucleotides of the sense strand (when hybridized to the antisense strand). There may be a gap of 1-6, 2-5 or 3-4 nucleotides between the polynucleotides of the sense strand. The sense strand may comprise at least three, at least four or at least five polynucleotides each of which is hybridized to the antisense strand. Preferably, there is a gap of one or more nucleotides between the adjacent polynucleotides of the sense strand (when hybridized to the antisense strand). There may be a gap of 1-6, 2-5 or 3-4 nucleotides between the adjacent polynucleotides of the sense strand. Preferably the polynucleotides of the sense strand are configured to enable the unaided delivery of the double-stranded nucleic acid molecule into a cell and the release of the antisense strand as a single-stranded molecule (e.g. following in-situ degradation of the sense strand).

Preferably, the configuration of the double-stranded nucleic acid molecule enables the delivery of the antisense strand into the nucleus of a cell as a single-stranded molecule.

Preferably one or more of the nucleotides of the 3′-overhang of the sense strand and/or one or more of the nucleotides of the 3′-overhang of the antisense strand is a therapeutic nucleotide analogue or a therapeutic nucleoside analogue.

The invention provides a double-stranded nucleic acid molecule comprising a sense strand and an antisense strand, wherein the sense strand has a 3′-overhang of at least three nucleotides (preferably from four to eight nucleotides) and the antisense strand has a 3′-overhang that is shorter than the 3′-overhang of the sense strand or the antisense strand does not have a 3′-overhang, and wherein one or more of the nucleotides of the 3′-overhang of the sense strand and/or the 3′-overhang of the antisense strand is a therapeutic nucleotide analogue or a therapeutic nucleoside analogue.

The 3′-overhang of the sense strand may comprise at least one, at least two, at least three, at least four or at least five therapeutic nucleotide analogue(s) and/or therapeutic nucleoside analogue(s). All of the nucleotides of the 3′-overhang of the sense strand may be therapeutic nucleotide analogue(s) and/or therapeutic nucleoside analogue(s)

The therapeutic nucleoside or nucleotide analogue may be a cytotoxic nucleoside or nucleotide analogue and/or an antiviral nucleoside or nucleotide analogue. Such analogues include adenosine analogues, deoxyadenosine analogues, cytidine analogues, deoxycytidine analogues, guanosine analogues, deoxyguanosine analogues, thymidine analogues, deoxythymidine analogues, uridine analogues and deoxyuridine analogues.

Examples of analogues include the following:

Adenosine analogues: BCX4430 (e.g. as a therapeutic against Ebola).

Deoxyadenosine analogues: Didanosine (ddl) (e.g. as a therapeutic against HIV); Vidarabine (e.g. for use in chemotherapy).

Deoxycytidine analogues: Cytarabine; Gemcitabine (e.g. for use in chemotherapy); Emtricitabine (FTC) (e.g. as a therapeutic against HIV); Lamivudine (3TC) (e.g. as a therapeutic against HIV, hepatitis B); Zalcitabine (ddC) (e.g. as a therapeutic against HIV).

Guanosine and deoxyguanosine analogues: Abacavir (e.g. as a therapeutic against HIV); Aciclovir; Entecavir (e.g. as a therapeutic against hepatitis B).

Thymidine and deoxythymidine analogues: Stavudine (d4T); Telbivudine (e.g. as a therapeutic against hepatitis B); Zidovudine (azidothymidine, or AZT) (e.g. as a therapeutic against HIV).

Deoxyuridine analogues: Idoxuridine; Trifluridine; 5-Fluorouracil (5FU).

Preferably the analogue is SFU. Further analogues and nucleotide modifications that can be used in accordance with the invention can be found in Table S1 in Jordheim et al., 2013 (Nature Rev. Drug Discovery 2013, 12, 447), the teaching therein with regard to such analogues and modifications is hereby incorporated by reference.

The double-stranded nucleic acid molecule may comprise a sense RNA strand and an antisense RNA strand, wherein the sense strand has a 3′-overhang of from 4 to 8 nucleotides, of which at least one nucleotide is a non-ribonucleotide (e.g. a deoxynucleotide), and wherein the antisense strand has a 3′-overhang that is shorter than said 3′-overhang of the sense strand.

The double-stranded nucleic acid molecule may comprise a sense RNA strand and an antisense RNA strand, wherein the sense strand has a 3′-overhang of from 4 to 8 nucleotides, of which at least one nucleotide is a non-ribonucleotide (e.g. a deoxynucleotide), and wherein said antisense strand has a 3′-overhang of 2 nucleotides.

The sense strand may have a 3′-overhang of at least 3 nucleotides, 4 to 8 nucleotides, 4 to 7 nucleotides, 4 to 6 nucleotides, 4 to 5 nucleotides, 5 to 8 nucleotides, 5 to 7 nucleotides, 5 to 6 nucleotides, 6 to 8 nucleotides, 6 to 7 nucleotides, or 7 to 8 nucleotides. Preferably, the sense strand has a 3′-overhang of 4, 5, 6, 7 or 8 nucleotides. Most preferably, the sense strand has a 3′-overhang of 5 nucleotides.

The antisense strand may have a 3′-overhang of at least one nucleotide, at least 2 nucleotides, 2 to 8 nucleotides, 2 to 7 nucleotides, 2 to 6 nucleotides, 2 to 5 nucleotides, 2 to 4 nucleotides, 2 to 3 nucleotides, 3 to 8 nucleotides, 3 to 7 nucleotides, 3 to 6 nucleotides, 3 to 5 nucleotides, 3 to 4 nucleotides, 4 to 8 nucleotides, 4 to 7 nucleotides, 4 to 6 nucleotides, 4 to 5 nucleotides, 5 to 8 nucleotides, 6 to 8 nucleotides, 6 to 7 nucleotides, or 7 to 8 nucleotides. Preferably, the antisense strand has a 3′-overhang of 2, 3, 4, 5, 6, 7 or 8 nucleotides. Most preferably, the antisense strand has a 3′-overhang of 2 nucleotides.

The antisense strand of the double-stranded nucleic acid molecule may not have a 3′-overhang but rather has a blunt end.

The skilled person will appreciate that the 3′-overhangs of the sense and antisense strands may be any combination of the lengths of overhang described above. Examples of preferred combinations are provided in Table 1 below:

TABLE 1

| Length of sense strand | Length of antisense strand |
|---|---|
| 3′ overhang (nucleotides) | 3′ overhang (nucleotides) |
| 4-8 | 2-5 |
| 4-8 | 2-4 |
| 4-8 | 2-3 |
| 4-8 | 2 |
| 5-8 | 2-5 |
| 5-8 | 2-4 |
| 5-8 | 2-3 |
| 5-8 | 2 |
| 4-7 | 2-5 |
| 4-7 | 2-4 |
| 4-7 | 2-3 |
| 4-7 | 2 |
| 5-7 | 2-5 |
| 5-7 | 2-4 |
| 5-7 | 2-3 |
| 5-7 | 2 |
| 4-6 | 2-5 |
| 4-6 | 2-4 |
| 4-6 | 2-3 |
| 4-6 | 2 |
| 5-6 | 2-5 |
| 5-6 | 2-4 |
| 5-6 | 2-3 |
| 5-6 | 2 |
| 4-5 | 2-4 |
| 4-5 | 2-3 |
| 4-5 | 2 |
| 5 | 2-5 |
| 5 | 2-4 |
| 5 | 2-3 |
| 5 | 2 |
| 6 | 2-5 |
| 6 | 2-4 |
| 6 | 2-3 |
| 6 | 2 |

Preferably, the sense strand has a 3′-overhang of from four to eight nucleotides and the antisense strand has a 3′-overhang of two nucleotides.

The sense strand may be an RNA strand and the antisense strand may be an RNA strand or a DNA strand. Alternatively, the sense strand may be a DNA strand and the antisense strand may be an RNA strand or a DNA strand. If the sense strand is an RNA strand, preferably all of the nucleotides of the sense strand in the double-stranded portion of the double-stranded nucleic acid molecule are ribonucleotides. If the sense strand is a DNA strand, preferably all of the nucleotides of the sense strand in the double-stranded portion of the double-stranded nucleic acid molecule are deoxynucleotides. If the antisense strand is an RNA strand, preferably all of the nucleotides of the antisense strand in the double-stranded portion of the double-stranded nucleic acid molecule are ribonucleotides. If the antisense strand is a DNA strand, preferably all of the nucleotides of the antisense strand in the double-stranded portion of the double-stranded nucleic acid molecule are deoxynucleotides.

The sense strand 3′-overhang and/or the antisense strand 3′-overhang may comprise one or more nucleotides that are not ribonucleotides (i.e. non-ribonucleotides). The sense strand 3′-overhang may comprise at least 1, 2, 3, 4, 5, 6, 7 or 8 non-ribonucleotides. Preferably, all of the nucleotides in the sense strand 3′-overhang are non-ribonucleotides. The antisense strand 3′-overhang may comprise at least 1, 2, 3, 4, 5, 6, 7 or 8 non-ribonucleotides. Preferably, all of the nucleotides in the antisense strand 3′-overhang are non-ribonucleotides.

The one or more non-ribonucleotides in the sense strand 3′-overhang and/or the antisense strand 3′ overhang may include one or more deoxynucleotides. The one or more deoxynucleotides may be any deoxynucleotide or combination of at least two deoxynucleotides, such as deoxythymidine (dT), deoxyadenosine (dA), deoxyguanosine (dG) or deoxycytosine (dC).

All of the nucleotides of the 3′-overhang of the sense and/or antisense strand may be deoxynucleotides. For example, all deoxynucleotides could be dT, all could be dA, all could be dG or all could be dC. Alternatively, all deoxynucleotides could be purine deoxynucleotides, i.e. dG and/or dA, or all could be pyrimidine deoxynucleotides, i.e. dT and/or dC. Alternatively, a mixture of purine and pyrimidine deoxynucleotides could be used, such as a mixture of dA, dG, dT and/or dC.

Examples of 3′-overhangs for the sense strand include: dT₄, dA₄, dG₄, dC₄, dT₅, dA₅, dG₅, dC₅, dT₆, dA₆, dG₆, dC₆, dT₇, dA₇, dG₇, dC₇, dT₈, dA₈, dG₈ and dC₈; preferably dT₅, dA₅, dG₅, dC₅, dT₆, dA₆, dG₆, dC₆, dT₇, dA₇, dG₇, dC₇, dT₈, dA₈, dG₈ and dC₈; preferably dT₅, dA₅, dG₅, dC₅, dT₆, dA₆, dG₆, dC₆, dT₇, dA₇, dG₇ and dC₇; preferably dT₅, dA₅, dG₅, dC₅, dT₆, dA₆, dG₆ and dC₆; or preferably dT₅, dA₅, dG₅ and dC₅. Alternatively, a combination of different deoxynucleotides may be included in the 3′-overhang of the sense strand.

Examples of 3′-overhangs for the antisense strand include: dT₂, dA₂, dG₂, dC₂, dT₃, dA₃, dG₃, dC₃, dT₄, dA₄, dG₄, dCₜ, dT₅, dA₅, dG₅ and dC₅; preferably dT₂, dA₂, dG₂, dC₂, dT₅, dA₅, dG₅ and dC₅; or preferably dT₂, dA₂, dG₂, dC₂. Alternatively, a combination of different deoxynucleotides may be included in the 3′-overhang of the antisense strand.

The skilled person will appreciate that the 3′-overhangs of the sense and antisense strands may be any combination of the overhangs described above. Examples of preferred combinations are provided in Table 2 below:

TABLE 2

| Sense strand 3′ overhang | Antisense strand 3′ overhang |
|---|---|
| dT_{5,} dA_{5,} dG₅ or dC₅ | dT₅, U₅, dT₂ or U₂ |
| dT_{5,} dA_{5,} dG₅ or dC₅ | dT₅ or U₅ |
| dT_{5,} dA_{5,} dG₅ or dC₅ | dT₂ or U₂ |
| dT_{5,} dA_{5,} dG₅ or dC₅ | dT₂ |
| dT_{5,} dA_{5,} dG₅ or dC₅ | No overhang |
| dT₅ or dA₅ | dT₅, U₅, dT₂ or U₂ |
| dT₅ or dA₅ | dT₅ or U₅ |
| dT₅ or dA₅ | dT₂ or U₂ |
| dT₅ or dA₅ | dT₂ |
| dT₅ or dA₅ | No overhang |
| dT₅ | dT₅, U₅, dT₂ or U₂ |
| dT₅ | dT₅ or U₅ |
| dT₅ | dT₂ or U₂ |
| dT₅ | dT₂ |
| dT₅ | No overhang |
| dT₅ or dA₅ | dT_{4,} dT₃, dT₂ or dT |
| dT₅ or dA₅ | dA_{4,} dA₃, dA₂ or dA |
| dT₅ or dA₅ | U₅, U_{4,} U₃, U₂ or U |
| dT₅ or dA₅ | dC_{4,} dC₃, dC₂ or dC |
| dT₅ or dA₅ | dG_{4,} dG₃, dG₂ or dG |

Alternatively, the one or more non-ribonucleotides in the 3′-overhang of the sense and/or antisense strand 3′ overhang may include one or more modified nucleotides, for example one or more dideoxynucleotides, or one or more other modified nucleotides. Modified nucleotides may have modifications on the ribose sugar, the phosphate backbone and/or nucleobase. Non-limiting examples of ribose modifications are analogues to modifications where 2′-OH is replaced by H, SH, SR, R, OR, Cl, Br, I, F, CN, NH₂, NHR, NR₂, guanidine, wherein R is an optionally substituted aryl group, C1-C6 alkyl, C2-C6-alkenyl or C2-C6 alkynyl group. Further nucleotide analogues and nucleotide modifications that can be used according to the embodiments are disclosed in paragraph [0017] of EP 1 407 044, the teaching therein with regard to such nucleotide analogues and modifications is hereby incorporated by reference. The double-stranded nucleic acid molecule may or may not comprise an orthoester-modified nucleotide.

One or more of the nucleotides of the 3′-overhang of the sense strand and/or antisense strand may be ribonucleotides. The ribonucleotide(s) may be riboadenosine (rA), riboguanosine (rG), ribouracil (rU) and/or ribocytosine (rC).

The nucleotides of the double-stranded nucleic acid molecule may or may not be modified with hydrophobic groups (e.g. cholesterol). The double-stranded nucleic acid molecule may or may not be conjugated to one or more other molecules.

The invention provides a double-stranded nucleic acid molecule comprising a sense strand and an antisense strand, wherein the sense strand and the antisense strand are linked together (by a linker) in a hairpin such that the sense strand is hybridized to the antisense strand, and wherein the double-stranded nucleic acid molecule comprises a 3′ overhang or a 5′ overhang of at least 3 nucleotides. Preferably the 3′ overhang or the 5′ overhang is 4-8 nucleotides.

The double-stranded nucleic acid molecule may comprise in the 5′ to 3′ direction the sense strand and then the antisense strand. Alternatively, the double-stranded nucleic acid molecule may comprise in the 5′ to 3′ direction the antisense strand and then the sense strand.

The sense strand and the antisense strand may be linked together by a single-stranded polynucleotide. The polynucleotide may comprise or consist of at least 3 nucleotides, 3-15 nucleotides, 4-14 nucleotides, 5-13 nucleotides, 6-12 nucleotides, 7-11 nucleotides or 8-10 nucleotides. The polynucleotide may form the loop of the hairpin.

The double-stranded nucleic acid molecule may comprise one or more cleavage sites between the sense strand and the antisense strand that enable cleavage of the linker to produce a double-stranded nucleic acid molecule of the invention having two 3′ ends. Preferably, cleavage occurs at the cleavage site(s) after delivery of the double-stranded nucleic acid molecule into a cell.

Preferably, the double-stranded nucleic acid molecule comprises in the 5′-3′ direction the antisense strand, a single-stranded polynucleotide, the sense strand and a 3′ overhang of 4-8 nucleotides.

The double-stranded nucleic acid molecule may be a short hairpin RNA (shRNA).

The double-stranded nucleic acid molecule may comprise at least two nucleotide mismatches between the sense and antisense strands. The mismatches may be any of the mismatches described herein.

The double-stranded portion of the double-stranded nucleic acid molecule may comprise a nick in the sense strand. The nick or nicks in the sense strand may be any of the types of nick described herein.

The sense strand of the double-stranded nucleic acid molecule may comprise two polynucleotides each of which is hybridized to the antisense strand. There may be a gap of one or more nucleotides between the polynucleotides of the sense strand. The two or more polynucleotides of the sense strand may be configured as described herein.

One or more of the nucleotides of the 3′-overhang or the 5′-overhang may be a therapeutic nucleotide analogue or a therapeutic nucleoside analogue. The therapeutic nucleoside or nucleotide analogue may be a cytotoxic nucleoside or nucleotide analogue and/or an antiviral nucleoside or nucleotide analogue. Other options for the analogue(s) are described herein.

The nucleotides of the 3′-overhang or the 5′-overhang may comprise or consist of any of the nucleotide options or sequences described herein in relation to the 3′-overhang of the sense strand or the 3′-overhang of the antisense strand.

The sense strand may be an RNA strand and the antisense strand may be an RNA strand or a DNA strand. Alternatively, the sense strand may be an DNA strand and the antisense strand may be an RNA strand or a DNA strand. If the sense strand is an RNA strand, preferably all of the nucleotides of the sense strand in the double-stranded portion of the double-stranded nucleic acid molecule are ribonucleotides. If the sense strand is a DNA strand, preferably all of the nucleotides of the sense strand in the double-stranded portion of the double-stranded nucleic acid molecule are deoxynucleotides. If the antisense strand is an RNA strand, preferably all of the nucleotides of the antisense strand in the double-stranded portion of the double-stranded nucleic acid molecule are ribonucleotides. If the antisense strand is a DNA strand, preferably all of the nucleotides of the antisense strand in the double-stranded portion of the double-stranded nucleic acid molecule are deoxynucleotides.

The double-stranded nucleic acid molecule may have a double-stranded portion with a length of at least 17 base pairs, at least 18 base pairs or preferably at least 19 base pairs. The double-stranded region may be 19 to 30 base pairs, 19 to 27 base pairs, 19 to 24 base pairs, or 19 to 21 base pairs. For example, the double-stranded region may be 19, 20, 21, 22, 23, 24, 25, 26, 27, 28 or 29 base pairs.

If the double-stranded nucleic acid molecule has a double-stranded portion of 19 base pairs, the sense strand preferably has a total length of 23 to 27 nucleotides. The antisense strand may then have a total length of 19 nucleotides if having a blunt end or preferably from 21 to 24 nucleotides, such as 21 nucleotides, with a 3′-overhang of two to five, such as two, nucleotides. Preferably, the double-stranded portion is 19 base pairs, the sense strand is 24 base pairs and the antisense strand is 21 base pairs.

The double-stranded portion of the nucleic acid molecule may or may not comprise one or more mismatches between a nucleotide on the sense strand and the opposite nucleotide on the antisense strand. Further options for mismatches are described herein.

The antisense strand of a double-stranded nucleic acid molecule of the invention may be a miRNA or an antisense oligonucleotide capable of gene silencing. The antisense oligonucleotide may be a miRNA inhibitor (anti-miR), an RNAase H-dependent antisense oligonucleotide, a Piwi-interacting RNA (piRNA) or an exon-skipping antisense oligonucleotide.

The double-stranded nucleic acid molecule of the invention may be a double-stranded small interfering ribonucleic acid (siRNA) molecule.

The antisense strand of a double-stranded nucleic acid molecule of the invention may be a CRISPR guide RNA.

The sense strand of the double-stranded nucleic acid molecule of the invention may comprise labels at the 5′-end for detection in analytical, in particular diagnostic purpose. The ‘label’ can be any chemical entity which enable the detection of the double-stranded nucleic acid molecule via, physical, chemical and/or biological means. The label may be a chromophore, a fluorophore and/or a radioactive molecule.

The invention further provides a double-stranded nucleic acid molecule of the invention for use as a medicament.

The invention further provides a double-stranded nucleic acid molecule of the invention for use in treating a disease by sequence-specific knockdown of a target RNA sequence, wherein at least a portion of an antisense strand of the double-stranded nucleic acid molecule has a nucleotide sequence that is complementary to a nucleotide sequence of the target RNA sequence.

The sequence of the antisense strand may be selected, in the double-stranded portion or at least a portion thereof, to be complementary to and capable of hybridizing to a target RNA or DNA sequence, preferably a target mRNA sequence. The sequence of the sense strand may be selected to be complementary to the antisense strand and form base pairs between the nucleotides in the respective strands in the double-stranded portion of the double-stranded nucleic acid molecule.

The sequence of the double-stranded nucleic acid molecule preferably has a sufficient identity to a target nucleotide sequence in order to mediate target-specific RNAi.

Preferably, the sequence of the double-stranded portion has an identity to the desired target nucleotide sequence of at least 50%, at least 70%, at least 85%, at least 90%, at least 95%, at least 96%, at least 97%, at least 98%, at least 99%, and most preferably 100%. Preferably, the sequence of the double-stranded portion has identity to the desired target nucleotide sequence over at least 10, 11, 12, 13, 14, 15, 16, 17, 18, 19, 20, 21, 22, 23, 24, 25, 26, 27, 28, 29 or 30 (contiguous) nucleotides.

The double-stranded nucleic acid molecule may be used to treat one or more of the diseases and/or disorders selected from genetic disorders, cancer (e.g. by silencing genes differentially upregulated in tumour cells and/or genes involved in cell division), HIV, other viral infections (e.g. infection caused by hepatitis A, hepatitis B, herpes simplex virus type 2, influenza, measles and/or respiratory syncytial virus), neurodegenerative diseases (e.g. Parkinson's disease and/or polyglutamine diseases such as Huntington's disease), ocular diseases (e.g. macular degeneration) and liver failure.

Potential antiviral therapies using the double-stranded nucleic acid molecule include one or more of the following: topical microbicide treatment to treat infection by herpes simplex virus type 2, inhibition of viral gene expression in cancerous cells, knockdown of host receptors and/or co-receptors for HIV, silencing of hepatitis A and/or hepatitis B genes, silencing of influenza gene expression, and inhibition of measles viral replication. Potential treatments for neurodegenerative diseases include treatment of polyglutamine diseases such as Huntington's disease.

A subject treated with the double-stranded nucleic acid molecule may receive the double-stranded nucleic acid molecule in combination with other forms of treatment for the disorder concerned, including treatment with drugs generally used for the treatment of the disorder. The drugs may be administered in one or several dosage units.

The double-stranded nucleic acid molecule is preferably administered to the subject as a pharmaceutical composition as described herein.

The invention further provides a cell transfection composition comprising a double-stranded nucleic acid molecule of the invention. The composition may not comprise an agent for facilitating release of the double-stranded nucleic acid molecule from the endosome into the cytosol of a cell. The composition may not comprise an agent for facilitating entry of the double-stranded nucleic acid molecule into a cell. The composition may not comprise a transfection reagent. The composition may not comprise a cationic agent. The composition may not comprise a cationic cell penetrating peptide (CPP); a cationic polymer or dendrimer e.g. polyethylenimine (PEI) or poly-D,L-lactide-co-glycolide (PLGA); and/or a cationic lipid (e.g. lipofectamine).

The cell transfection composition may or may not comprise a carrier e.g. an agent or a formulation. Preferably, the carrier promotes accumulation of the double-stranded nucleic acid molecule at a target site and/or protects the double-stranded nucleic acid molecule from undesirable interactions with biological milieu components and/or protects the double-stranded nucleic acid molecule from metabolism and/or degradation.

The carrier may be a viral carrier or a non-viral carrier. Viral carriers include a lentiviral vector or an adenoviral vector for delivery of a DNA-based construct encoding the double-stranded nucleic acid molecule. Non-viral carriers (or vectors) include complexing the double-stranded nucleic acid molecule with a cationic agent such as a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine). Preferably, the carrier is not a cationic agent. Preferably the carrier is not a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine).

The carrier may be a small molecule (e.g., cholesterol, bile acid, and/or lipid), polymer, protein (e.g. an antibody), and/or aptamer (e.g. RNA) that is conjugated to the double-stranded nucleic acid molecule. The carrier may be a nanoparticulate formulation used to encapsulate the double-stranded nucleic acid molecule.

The carrier may be a modification of the double-stranded nucleic acid molecule with a targeting ligand (e.g. an antibody), a peptide, a small molecule (e.g. folic acid and/or biotin), or a polymer present in extracellular matrix (e.g. hyaluronic acid and/or chondroitin sulphate), or a hydrophobic modification of the double-stranded nucleic acid molecule (e.g. using cholesterol and/or α-tocopherol). Preferably, the carrier is not a hydrophobic modification of the double-stranded nucleic acid molecule (e.g. using cholesterol and/or α-tocopherol).

The cell transfection composition may or may not comprise an agent selected from a photosensitizing agent and/or a radical initiator e.g. a photoinitiator. Preferably, this agent improves the function of the double-stranded nucleic acid molecule at a target site (e.g. enhances the knockdown of a target RNA sequence as described herein) and/or protects the double-stranded nucleic acid molecule from metabolism and/or degradation.

The invention further provides a pharmaceutical composition comprising a double-stranded nucleic acid molecule of the invention and a pharmaceutically acceptable diluent. The pharmaceutical composition may not comprise an agent for facilitating release of the double-stranded nucleic acid molecule from the endosome into the cytosol of a cell. The pharmaceutical composition may not comprise an agent for facilitating entry of the double-stranded nucleic acid molecule into a cell. The pharmaceutical composition may not comprise a cationic agent. The pharmaceutical composition may not comprise a cationic cell penetrating peptide (CPP); a cationic polymer or dendrimer e.g. polyethylenimine (PEI) or poly-D,L-lactide-co-glycolide (PLGA); and/or a cationic lipid (e.g. lipofectamine).

The pharmaceutical composition may or may not comprise a carrier e.g. an agent or a formulation. Preferably, the carrier promotes accumulation of the double-stranded nucleic acid molecule at a target site and/or protects the double-stranded nucleic acid molecule from undesirable interactions with biological milieu components and/or protects the double-stranded nucleic acid molecule from metabolism and/or degradation.

The carrier may be a viral carrier or a non-viral carrier. Viral carriers include a lentiviral vector or an adenoviral vector for delivery of a DNA-based construct encoding the double-stranded nucleic acid molecule. Non-viral carriers (or vectors) include complexing the double-stranded nucleic acid molecule with a cationic agent such as a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine). Preferably, the carrier is not a cationic agent. Preferably the carrier is not a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine).

The carrier may be a small molecule (e.g., cholesterol, bile acid, and/or lipid), polymer, protein (e.g. an antibody), and/or aptamer (e.g. RNA) that is conjugated to the double-stranded nucleic acid molecule. The carrier may be a nanoparticulate formulation used to encapsulate the double-stranded nucleic acid molecule.

The carrier may be a modification of the double-stranded nucleic acid molecule with a targeting ligand (e.g. an antibody), a peptide, a small molecule (e.g. folic acid and/or biotin), or a polymer present in extracellular matrix (e.g. hyaluronic acid and/or chondroitin sulphate), or a hydrophobic modification of the double-stranded nucleic acid molecule (e.g. using cholesterol and/or α-tocopherol). Preferably, the carrier is not a hydrophobic modification of the double-stranded nucleic acid molecule (e.g. using cholesterol and/or α-tocopherol).

The pharmaceutically acceptable diluent may be saline, a buffered solution (e.g. a buffered aqueous solution) or another excipient.

The pharmaceutical composition may be in form of a solution, e.g. an injectable solution, a cream, ointment, tablet, suspension or the like. The composition may be administered in any suitable way, e.g. by injection, by oral, topical, nasal, rectal application etc.

The pharmaceutical composition may or may not comprise an agent selected from a photosensitizing agent and/or a radical initiator e.g. a photoinitiator. Preferably, this agent improves the function of the double-stranded nucleic acid molecule at a target site (e.g. enhances the knockdown of a target RNA sequence as described herein) and/or protects the double-stranded nucleic acid molecule from metabolism and/or degradation.

The invention further provides a cell transfection method comprising contacting (in vitro) a cell to be transfected with a double-stranded nucleic acid molecule of the invention, and wherein the double-stranded nucleic acid molecule is transfected into the cytosol of the cell.

The step of contacting (in vitro) may comprise contacting the cell to be transfected by the double-stranded nucleic acid molecule in the absence of a transfection agent. The step of contacting (in vitro) may comprise contacting the cell to be transfected by the double-stranded nucleic acid molecule in the absence of a transfection reagent. The step of contacting (in vitro) the cell to be transfected with a double-stranded nucleic acid molecule may be performed in the absence of an agent for facilitating release of the double-stranded nucleic acid molecule from the endosome into the cytosol of said cell. The step of contacting (in vitro) the cell to be transfected with a double-stranded nucleic acid molecule may be performed in the absence of an agent for facilitating entry of the double-stranded nucleic acid molecule into the cell. The step of contacting (in vitro) the cell to be transfected with a double-stranded nucleic acid molecule may be performed in the absence of a cationic agent. The step of contacting (in vitro) the cell to be transfected with a double-stranded nucleic acid molecule may be performed in the absence of a cationic cell penetrating peptide (CPP); a cationic polymer or dendrimer e.g. polyethylenimine (PEI) or poly-D,L-lactide-co-glycolide (PLGA); and/or a cationic lipid (e.g. lipofectamine).

The cell may be contacted with the double-stranded nucleic acid molecule in the presence or absence of a carrier e.g. an agent or a formulation. Preferably, the carrier promotes accumulation of the double-stranded nucleic acid molecule at a target site and/or protects the double-stranded nucleic acid molecule from undesirable interactions with biological milieu components and/or protects the double-stranded nucleic acid molecule from metabolism and/or degradation.

The carrier may be a viral carrier or a non-viral carrier. Viral carriers include a lentiviral vector or an adenoviral vector for delivery of a DNA-based construct encoding the double-stranded nucleic acid molecule. Non-viral carriers (or vectors) include complexing the double-stranded nucleic acid molecule with a cationic agent such as a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine). Preferably, the carrier is not a cationic agent. Preferably the carrier is not a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine).

The carrier may be a small molecule (e.g., cholesterol, bile acid, and/or lipid), polymer, protein (e.g. an antibody), and/or aptamer (e.g. RNA) that is conjugated to the double-stranded nucleic acid molecule. The carrier may be a nanoparticulate formulation used to encapsulate the double-stranded nucleic acid molecule.

The carrier may be a modification of the double-stranded nucleic acid molecule with a targeting ligand (e.g. an antibody), a peptide, a small molecule (e.g. folic acid and/or biotin), or a polymer present in extracellular matrix (e.g. hyaluronic acid and/or chondroitin sulphate), or a hydrophobic modification of the double-stranded nucleic acid molecule (e.g. using cholesterol and/or α-tocopherol). Preferably, the carrier is not a hydrophobic modification of the double-stranded nucleic acid molecule (e.g. using cholesterol and/or α-tocopherol).

The step of contacting (in vitro) may comprise contacting the cell to be transfected by the double-stranded nucleic acid molecule without the use of direction injection, electroporation, sonoporation, optical transfection, protoplast fusion, impalefection, hydrodynamic delivery, magnetofection, particle bombardment and/or nucleofection.

The cell or cells to be transfected may be provided in a cell culture medium (e.g. in a Petri dish, culture vessel or well, etc). The double-stranded nucleic acid molecule may be added directly to the cell culture medium or the cells may be added to a solution, such as saline, a buffered solution or a cell culture medium, comprising the double-stranded nucleic acid molecule.

After cellular delivery of a double-stranded nucleic acid molecule of the invention, the antisense strand may be released in the cell as a single-stranded nucleic acid molecule.

The cell may be contacted with the double-stranded nucleic acid molecule in the presence or absence of an agent selected from a photosensitizing agent and/or a radical initiator e.g. a photoinitiator. Preferably, this agent improves the function of the double-stranded nucleic acid molecule at a target site (e.g. enhances the knockdown of a target RNA sequence as described herein) and/or protects the double-stranded nucleic acid molecule from metabolism and/or degradation.

The invention further provides a cell obtainable by the methods of the invention. Thus, the cell may comprise a double-stranded nucleic acid molecule of the invention.

The invention further provides a cell transfected with a double-stranded nucleic acid molecule of the invention or a nucleotide sequence encoding the double-stranded nucleic acid molecule.

The invention further provides a method of target-specific ribonucleic acid (RNA) interference in a cell comprising contacting (e.g. in vitro) the cell with a double-stranded nucleic acid molecule of the invention, wherein at least at least a portion of the antisense strand of the double stranded nucleic acid molecule has a nucleotide sequence that is complementary to a nucleotide sequence of a target RNA sequence.

The step of contacting (e.g. in vitro) the cell with a double-stranded nucleic acid molecule may be performed in the absence of an agent for facilitating release of said double-stranded nucleic acid molecule from the endosome into the cytosol of said cell. The step of contacting (e.g. in vitro) the cell with a double-stranded nucleic acid molecule may be performed in the absence of an agent for facilitating entry of the double-stranded nucleic acid molecule into the cell. The step of contacting (e.g. in vitro) the cell with a double-stranded nucleic acid molecule may be performed in the absence of a cationic agent. The step of contacting (e.g. in vitro) the cell with a double-stranded nucleic acid molecule may be performed in the absence of a cationic cell penetrating peptide (CPP); a cationic polymer or dendrimer e.g. polyethylenimine (PEI) or poly-D,L-lactide-co-glycolide (PLGA); and/or a cationic lipid (e.g. lipofectamine).

The cell may be an animal cell, such as mammal cell (e.g. a human cell), a fungal cell, a cell of a micro-organism (e.g. a prokaryotic cell or a eukaryotic cell), or a plant cell.

The step of contacting the cell with a double-stranded nucleic acid molecule may be performed in vivo. For example, the double-stranded nucleic acid molecule may be administered to an organism in which RNAi and gene knockdown is desired. The organism may be an animal, such as mammal (e.g. a human), a fungus, a micro-organism, or a plant.

The sequence of the antisense strand of the double-stranded nucleic acid molecule may be selected, in the double-stranded portion or at least a portion thereof, to be complementary to and capable of hybridizing to a target RNA or DNA sequence, preferably a target mRNA sequence. The sequence of the sense strand may be selected to be complementary to the antisense strand and form base pairs between the nucleotides in the respective strands in the double-stranded portion of the double-stranded nucleic acid molecule.

The sequence of the double-stranded nucleic acid molecule preferably has a sufficient identity to a target nucleotide sequence in order to mediate target-specific RNAi.

Preferably, the sequence of the double-stranded portion has an identity to the desired target nucleotide sequence of at least 50%, at least 70%, at least 85%, at least 90%, at least 95%, at least 96%, at least 97%, at least 98%, at least 99%, and most preferably 100%. Preferably, the sequence of the double-stranded portion has identity to the desired target nucleotide sequence over at least 10, 11, 12, 13, 14, 15, 16, 17, 18, 19, 20, 21, 22, 23, 24, 25, 26, 27, 28, 29 or 30 (contiguous) nucleotides.

The cell may be contacted with the double-stranded nucleic acid molecule in the presence or absence of a carrier e.g. an agent or a formulation. Preferably, the carrier promotes accumulation of the double-stranded nucleic acid molecule at a target site and/or protects the double-stranded nucleic acid molecule from undesirable interactions with biological milieu components and/or protects the double-stranded nucleic acid molecule from metabolism and/or degradation.

The carrier may be a viral carrier or a non-viral carrier. Viral carriers include a lentiviral vector or an adenoviral vector for delivery of a DNA-based construct encoding the double-stranded nucleic acid molecule. Non-viral carriers (or vectors) include complexing the double-stranded nucleic acid molecule with a cationic agent such as a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine). Preferably, the carrier is not a cationic agent. Preferably the carrier is not a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine).

The carrier may be a small molecule (e.g., cholesterol, bile acid, and/or lipid), polymer, protein (e.g. an antibody), and/or aptamer (e.g. RNA) that is conjugated to the double-stranded nucleic acid molecule. The carrier may be a nanoparticulate formulation used to encapsulate the double-stranded nucleic acid molecule.

The carrier may be a modification of the double-stranded nucleic acid molecule with a targeting ligand (e.g. an antibody), a peptide, a small molecule (e.g. folic acid and/or biotin), or a polymer present in extracellular matrix (e.g. hyaluronic acid and/or chondroitin sulphate), or a hydrophobic modification of the double-stranded nucleic acid molecule (e.g. using cholesterol and/or α-tocopherol). Preferably, the carrier is not a hydrophobic modification of the double-stranded nucleic acid molecule (e.g. using cholesterol and/or α-tocopherol).

The cell may be contacted with the double-stranded nucleic acid molecule in the presence or absence of an agent selected from a photosensitizing agent and/or a radical initiator e.g. a photoinitiator. Preferably, this agent improves the function of the double-stranded nucleic acid molecule at a target site (e.g. enhances the knockdown of a target RNA sequence as described herein) and/or protects the double-stranded nucleic acid molecule from metabolism and/or degradation.

The invention further provides the use of a double-stranded nucleic acid molecule of the invention for target-specific ribonucleic acid (RNA) interference in a cell (e.g. in vitro), wherein the 3′-overhang facilitates release of the double-stranded nucleic acid molecule from the endosome into the cytosol of the cell.

The use of a double-stranded nucleic acid molecule for target-specific ribonucleic acid (RNA) interference in a cell (e.g. in vitro) may be in the absence of an agent for facilitating release of said double-stranded nucleic acid molecule from the endosome into the cytosol of said cell. The use of a double-stranded nucleic acid molecule for target-specific ribonucleic acid (RNA) interference in a cell (e.g. in vitro) may be in the absence of an agent for facilitating entry of the double-stranded nucleic acid molecule into the cell. The use of a double-stranded nucleic acid molecule for target-specific ribonucleic acid (RNA) interference in a cell (e.g. in vitro) may be in the absence of a cationic agent. The use of a double-stranded nucleic acid molecule for target-specific ribonucleic acid (RNA) interference in a cell (e.g. in vitro) may be in the absence of a cationic cell penetrating peptide (CPP); a cationic polymer or dendrimer e.g. polyethylenimine (PEI) or poly-D,L-lactide-co-glycolide (PLGA); and/or a cationic lipid (e.g. lipofectamine).

The cell may be an animal cell, such as mammal cell (e.g. a human cell), a fungal cell, a cell of a micro-organism (e.g. a prokaryotic cell or a eukaryotic cell), or a plant cell.

The use of a double-stranded nucleic acid molecule of the invention may be for target-specific ribonucleic acid (RNA) interference in a cell in vivo. For example, in an organism in which RNAi and gene knockdown is desired. The organism may be an animal, such as mammal (e.g. a human), a fungus, a micro-organism, or a plant.

In order to mediate target-specific ribonucleic acid (RNA) interference in a cell at least at least a portion of the antisense strand of the double stranded nucleic acid molecule has a nucleotide sequence that is complementary to a nucleotide sequence of a target RNA sequence.

The sequence of the antisense strand is selected, in the double-stranded portion or at least a portion thereof, to be complementary to and capable of hybridizing to a target RNA or DNA sequence, preferably a target mRNA sequence. The sequence of the sense strand is selected to be complementary to the antisense strand and form base pairs between the nucleotides in the respective strands in the double-stranded portion of the double-stranded nucleic acid molecule.

The sequence of the double-stranded nucleic acid molecule preferably has a sufficient identity to a target nucleotide sequence in order to mediate target-specific RNAi.

Preferably, the sequence of the double-stranded portion has an identity to the desired target nucleotide sequence of at least 50%, at least 70%, at least 85%, at least 90%, at least 95%, at least 96%, at least 97%, at least 98%, at least 99%, and most preferably 100%. Preferably, the sequence of the double-stranded portion has identity to the desired target nucleotide sequence over at least 10, 11, 12, 13, 14, 15, 16, 17, 18, 19, 20, 21, 22, 23, 24, 25, 26, 27, 28, 29 or 30 (contiguous) nucleotides.

The double-stranded nucleic acid molecule may be used with or without a carrier e.g. an agent or a formulation. Preferably, the carrier promotes accumulation of the double-stranded nucleic acid molecule at a target site and/or protects the double-stranded nucleic acid molecule from undesirable interactions with biological milieu components and/or protects the double-stranded nucleic acid molecule from metabolism and/or degradation.

The carrier may be a viral carrier or a non-viral carrier. Viral carriers include a lentiviral vector or an adenoviral vector for delivery of a DNA-based construct encoding the double-stranded nucleic acid molecule. Non-viral carriers (or vectors) include complexing the double-stranded nucleic acid molecule with a cationic agent such as a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine). Preferably, the carrier is not a cationic agent. Preferably the carrier is not a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine).

The carrier may be a small molecule (e.g., cholesterol, bile acid, and/or lipid), polymer, protein (e.g. an antibody), and/or aptamer (e.g. RNA) that is conjugated to the double-stranded nucleic acid molecule. The carrier may be a nanoparticulate formulation used to encapsulate the double-stranded nucleic acid molecule.

The carrier may be a modification of the double-stranded nucleic acid molecule with a targeting ligand (e.g. an antibody), a peptide, a small molecule (e.g. folic acid and/or biotin), or a polymer present in extracellular matrix (e.g. hyaluronic acid and/or chondroitin sulphate), or a hydrophobic modification of the double-stranded nucleic acid molecule (e.g. using cholesterol and/or α-tocopherol). Preferably, the carrier is not a hydrophobic modification of the double-stranded nucleic acid molecule (e.g. using cholesterol and/or α-tocopherol).

After cellular delivery of a double-stranded nucleic acid molecule of the invention, the antisense strand may be released in the cell as a single-stranded nucleic acid molecule.

The double-stranded nucleic acid molecule may be used in the presence or absence of an agent selected from a photosensitizing agent and/or a radical initiator e.g. a photoinitiator. Preferably, this agent improves the function of the double-stranded nucleic acid molecule at a target site (e.g. enhances the knockdown of a target RNA sequence as described herein) and/or protects the double-stranded nucleic acid molecule from metabolism and/or degradation.

The invention further provides a double-stranded nucleic acid molecule of the invention for use in the treatment of a disease by sequence-specific knockdown of a target RNA sequence, wherein at least a portion of the antisense strand of the double-stranded nucleic acid molecule has a nucleotide sequence that is complementary to a nucleotide sequence of the target RNA sequence.

The double-stranded nucleic acid molecule may be administered to the subject in the absence of an agent for facilitating release of said double-stranded nucleic acid molecule from the endosome into the cytosol of a cell of the subject. The double-stranded nucleic acid molecule may be administered to the subject in the absence of an agent for facilitating entry of the double-stranded nucleic acid molecule into a cell of the subject. The double-stranded nucleic acid molecule may be administered to the subject in the absence of a cationic agent. The double-stranded nucleic acid molecule may be administered to the subject in the absence of a cationic cell penetrating peptide (CPP); a cationic polymer or dendrimer e.g. polyethylenimine (PEI) or poly-D,L-lactide-co-glycolide (PLGA); and/or a cationic lipid (e.g. lipofectamine).

The double-stranded nucleic acid molecule is preferably administered to the subject as a pharmaceutical composition as described herein.

The sequence of the antisense strand is selected, in the double-stranded portion or at least a portion thereof, to be complementary to and capable of hybridizing to a target RNA or DNA sequence, preferably a target mRNA sequence. The sequence of the sense strand is selected to be complementary to the antisense strand and form base pairs between the nucleotides in the respective strands in the double-stranded portion of the double-stranded nucleic acid molecule.

The sequence of the double-stranded nucleic acid molecule preferably has a sufficient identity to a target nucleotide sequence in order to mediate target-specific RNAi. Preferably, the sequence of the double-stranded portion has an identity to the desired target nucleotide sequence of at least 50%, at least 70%, at least 85%, at least 90%, at least 95%, at least 96%, at least 97%, at least 98%, at least 99%, and most preferably 100%. Preferably, the sequence of the double-stranded portion has identity to the desired target nucleotide sequence over at least 10, 11, 12, 13, 14, 15, 16, 17, 18, 19, 20, 21, 22, 23, 24, 25, 26, 27, 28, 29 or 30 (contiguous) nucleotides.

The double-stranded nucleic acid molecule may be administered with or without a carrier e.g. an agent or a formulation. Preferably, the carrier promotes accumulation of the double-stranded nucleic acid molecule at a target site and/or protects the double-stranded nucleic acid molecule from undesirable interactions with biological milieu components and/or protects the double-stranded nucleic acid molecule from metabolism and/or degradation.

The carrier may be a viral carrier or a non-viral carrier. Viral carriers include a lentiviral vector or an adenoviral vector for delivery of a DNA-based construct encoding the double-stranded nucleic acid molecule. Non-viral carriers (or vectors) include complexing the double-stranded nucleic acid molecule with a cationic agent such as a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine). Preferably, the carrier is not a cationic agent. Preferably the carrier is not a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine).

The carrier may be a small molecule (e.g., cholesterol, bile acid, and/or lipid), polymer, protein (e.g. an antibody), and/or aptamer (e.g. RNA) that is conjugated to the double-stranded nucleic acid molecule. The carrier may be a nanoparticulate formulation used to encapsulate the double-stranded nucleic acid molecule.

The carrier may be a modification of the double-stranded nucleic acid molecule with a targeting ligand (e.g. an antibody), a peptide, a small molecule (e.g. folic acid and/or biotin), or a polymer present in extracellular matrix (e.g. hyaluronic acid and/or chondroitin sulphate), or a hydrophobic modification of the double-stranded nucleic acid molecule (e.g. using cholesterol and/or α-tocopherol). Preferably, the carrier is not a hydrophobic modification of the double-stranded nucleic acid molecule (e.g. using cholesterol and/or α-tocopherol).

The double-stranded nucleic acid molecule may be administered with or without an agent selected from a photosensitizing agent and/or a radical initiator e.g. a photoinitiator. Preferably, this agent improves the function of the double-stranded nucleic acid molecule at a target site (e.g. enhances the knockdown of a target RNA sequence as described herein) and/or protects the double-stranded nucleic acid molecule from metabolism and/or degradation.

The double-stranded nucleic acid molecule may be used to treat one or more of the diseases and/or disorders selected from genetic disorders, cancer (e.g. by silencing genes differentially upregulated in tumour cells and/or genes involved in cell division), HIV, other viral infections (e.g. infection caused by hepatitis A, hepatitis B, herpes simplex virus type 2, influenza, measles and/or respiratory syncytial virus), neurodegenerative diseases (e.g. Parkinson's disease and/or polyglutamine diseases such as Huntington's disease), ocular diseases (e.g. macular degeneration) and liver failure.

Potential antiviral therapies using the double-stranded nucleic acid molecule include one or more of the following: topical microbicide treatment to treat infection by herpes simplex virus type 2, inhibition of viral gene expression in cancerous cells, knockdown of host receptors and/or co-receptors for HIV, silencing of hepatitis A and/or hepatitis B genes, silencing of influenza gene expression, and inhibition of measles viral replication. Potential treatments for neurodegenerative diseases include treatment of polyglutamine diseases such as Huntington's disease.

A subject treated with the double-stranded nucleic acid molecule may receive the double-stranded nucleic acid molecule in combination with other forms of treatment for the disorder concerned, including treatment with drugs generally used for the treatment of the disorder. The drugs may be administered in one or several dosage units.

The invention further provides a method of treating a patient suffering from a disease comprising administering a double-stranded nucleic acid molecule of the invention to the patient, wherein the double-stranded nucleic acid molecule provides sequence-specific knockdown of a target RNA sequence in the patient, and wherein at least a portion of an antisense strand of the double-stranded nucleic acid molecule has a nucleotide sequence that is complementary to a nucleotide sequence of the target RNA sequence.

The double-stranded nucleic acid molecule may be administered to the patient in the absence of an agent for facilitating release of the double-stranded nucleic acid from the endosome into the cytosol of a cell of said patient. The double-stranded nucleic acid molecule may be administered to the patient in the absence of an agent for facilitating entry of the double-stranded nucleic acid molecule into a cell of the patient. The double-stranded nucleic acid molecule may be administered to the patient in the absence of a cationic agent. The double-stranded nucleic acid molecule may be administered to the patient in the absence of a cationic cell penetrating peptide (CPP); a cationic polymer or dendrimer e.g. polyethylenimine (PEI) or poly-D,L-lactide-co-glycolide (PLGA); and/or a cationic lipid (e.g. lipofectamine).

The double-stranded nucleic acid molecule is preferably administered to the patient as a pharmaceutical composition as described herein.

The sequence of the antisense strand is selected, in the double-stranded portion or at least a portion thereof, to be complementary to and capable of hybridizing to a target RNA or DNA sequence, preferably a target mRNA sequence. The sequence of the sense strand is selected to be complementary to the antisense strand and form base pairs between the nucleotides in the respective strands in the double-stranded portion of the double-stranded nucleic acid molecule.

The sequence of the double-stranded nucleic acid molecule preferably has a sufficient identity to a target nucleotide sequence in order to mediate target-specific RNAi. Preferably, the sequence of the double-stranded portion has an identity to the desired target nucleotide sequence of at least 50%, at least 70%, at least 85%, at least 90%, at least 95%, at least 96%, at least 97%, at least 98%, at least 99%, and most preferably 100%. Preferably, the sequence of the double-stranded portion has identity to the desired target nucleotide sequence over at least 10, 11, 12, 13, 14, 15, 16, 17, 18, 19, 20, 21, 22, 23, 24, 25, 26, 27, 28, 29 or 30 (contiguous) nucleotides.

The double-stranded nucleic acid molecule may be administered to the patient with or without a carrier e.g. an agent or a formulation. Preferably, the carrier promotes accumulation of the double-stranded nucleic acid molecule at a target site and/or protects the double-stranded nucleic acid molecule from undesirable interactions with biological milieu components and/or protects the double-stranded nucleic acid molecule from metabolism and/or degradation.

The carrier may be a viral carrier or a non-viral carrier. Viral carriers include a lentiviral vector or an adenoviral vector for delivery of a DNA-based construct encoding the double-stranded nucleic acid molecule. Non-viral carriers (or vectors) include complexing the double-stranded nucleic acid molecule with a cationic agent such as a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine). Preferably, the carrier is not a cationic agent. Preferably the carrier is not a cationic cell penetrating peptide (CPP); a cationic polymer or dendrimer e.g. polyethylenimine (PEI) and poly-D,L-lactide-co-glycolide (PLGA); and/or a cationic lipid (e.g. lipofectamine).

The carrier may be a small molecule (e.g., cholesterol, bile acid, and/or lipid), polymer, protein (e.g. an antibody), and/or aptamer (e.g. RNA) that is conjugated to the double-stranded nucleic acid molecule. The carrier may be a nanoparticulate formulation used to encapsulate the double-stranded nucleic acid molecule.

The carrier may be a modification of the double-stranded nucleic acid molecule with a targeting ligand (e.g. an antibody), a peptide, a small molecule (e.g. folic acid and/or biotin), or a polymer present in extracellular matrix (e.g. hyaluronic acid and/or chondroitin sulphate), or a hydrophobic modification of the double-stranded nucleic acid molecule (e.g. using cholesterol and/or α-tocopherol). Preferably, the carrier is not a hydrophobic modification of the double-stranded nucleic acid molecule (e.g. using cholesterol and/or α-tocopherol).

The double-stranded nucleic acid molecule may be administered to the patient with or without an agent selected from a photosensitizing agent and/or a radical initiator e.g. a photoinitiator. Preferably, this agent improves the function of the double-stranded nucleic acid molecule at a target site (e.g. enhances the knockdown of a target RNA sequence as described herein) and/or protects the double-stranded nucleic acid molecule from metabolism and/or degradation.

The double-stranded nucleic acid molecule may be used to treat one or more of the diseases and/or disorders selected from genetic disorders, cancer (e.g. by silencing genes differentially upregulated in tumour cells and/or genes involved in cell division), HIV, other viral infections (e.g. infection caused by hepatitis A, hepatitis B, herpes simplex virus type 2, influenza, measles and/or respiratory syncytial virus), neurodegenerative diseases (e.g. Parkinson's disease and/or polyglutamine diseases such as Huntington's disease), ocular diseases (e.g. macular degeneration) and liver failure.

Potential antiviral therapies using the double-stranded nucleic acid molecule include one or more of the following: topical microbicide treatment to treat infection by herpes simplex virus type 2, inhibition of viral gene expression in cancerous cells, knockdown of host receptors and/or co-receptors for HIV, silencing of hepatitis A and/or hepatitis B genes, silencing of influenza gene expression, and inhibition of measles viral replication. Potential treatments for neurodegenerative diseases include treatment of polyglutamine diseases such as Huntington's disease.

A subject treated with the double-stranded nucleic acid molecule may receive the double-stranded nucleic acid molecule in combination with other forms of treatment for the disorder concerned, including treatment with drugs generally used for the treatment of the disorder. The drugs may be administered in one or several dosage units.

The double-stranded nucleic acid molecules of the invention have improved characteristics in terms of cellular uptake, endosomal escape and/or increased gene silencing or knockdown activity.

The double-stranded nucleic acid molecules of the invention may mediate sequence-specific knockdown of a target RNA sequence. The % knockdown of the target RNA sequence may be at least 20%, at least 30%, at least 40%, at least 50%, at least 60%, at least 70%, at least 75%, at least 80%, at least 85%, at least 90%, or at least 95%, when compared to the normal level of expression of the target RNA sequence. The % knockdown of the target RNA sequence by a double-stranded nucleic acid molecule of the invention may be at least 1.5 times, at least 1.75 times, at least 2 times, at least 2.25 times, at least 2.5 times, at least 2.75 times, at least 3 times, at least 3.25 times, at least 3.5 times, at least 3.75 times, or at least 4 times the level of the knockdown achieved by a canonical double stranded nucleic acid molecule e.g. an siRNA molecule having a double-stranded region identical to the double-stranded region of the double-stranded nucleic acid of the invention with a sense strand 3′ overhang of 2 nucleotides and an antisense strand 3′ overhang of 2 nucleotides (e.g. a sense strand 3′ overhang of dT₂ and an antisense strand 3′ overhang of dT₂). The % knockdown of a target RNA may be determined by Real-Time PCR (RT-PCR).

The invention further provides a kit comprising a double-stranded nucleic acid molecule or composition of the invention. The kit may be suitable or intended for performing a method of the invention. The kit may further comprise one or more additional agents or reagents for performing one or more of the steps of the methods of the invention.

The invention further provides a method of preparing a double-stranded nucleic acid molecule of the embodiments. The method generally comprises synthesizing a sense strand and an antisense strand as described herein. The sense strand and the antisense strand are capable of hybridizing in a double-stranded portion of the double-stranded nucleic acid molecule. The method preferably also comprises combining the sense strand and the antisense strand under conditions allowing hybridization for form the double-stranded nucleic acid molecule.

Methods of synthesizing RNA and DNA strands are well known in the art, including for instance phosphoramidite chemistry, H-phosphonate chemistry and enzymatic chain extension. The sense and antisense strands can also be prepared by enzymatic transcription from DNA templates.

### BRIEF DESCRIPTION OF THE DRAWINGS

The embodiments, together with further objects and advantages thereof, may best be understood by making reference to the following description taken together with the accompanying drawings, in which:

FIGS. 1A-1F illustrate different double-stranded nucleic acid molecules 100, 200, 300, 400, 500 and 600. In FIG. 1A, the double-stranded nucleic acid molecule 100 has a sense strand 110 with a 3′-overhang 115 of four to eight nucleotides and an antisense strand 120 with a 3′-overhang 125 of two nucleotides. FIG. 1B illustrates a double-stranded nucleic acid molecule 200 where the sense strand 210 has a four to eight nucleotide 3′-overhang 215 and the antisense strand 220 has a 3′-overhang of two to five nucleotides 225. FIG. 1C illustrates a double-stranded nucleic acid molecule 300 in which the antisense strand 320 has a blunt end and the sense strand 310 has a four to eight nucleotide 3′-overhang 315. FIG. 1D illustrates a double-stranded nucleic acid molecule 400 in which the sense strand 410 is comprised of two polynucleotides hybridized to the antisense strand 420 forming a double-stranded portion 430. The two polynucleotides of the sense strand 410 are separated by a nick 416. The sense strand 410 has a four to eight nucleotide 3′-overhang 415 and the antisense strand 420 has 3′-overhang of zero to five nucleotides 425. FIG. 1E illustrates a double-stranded nucleic acid molecule 500 in which the sense strand 510 and the antisense strand 520 form a double-stranded portion 530. The sense strand has a mismatch 516. The sense strand 510 has a four to eight nucleotide 3′-overhang 515 and the antisense strand 520 has 3′-overhang of zero to five nucleotides 525. FIG. 1F illustrates a double-stranded nucleic acid molecule 600 in which the sense strand 610 and the antisense strand 620 are linked together as a hairpin such that the sense strand 610 and antisense strand 620 form a double-stranded portion 630. The molecule has a 3′-overhang of four to eight nucleotides 615.

In FIGS. 1A-1F, reference numbers 130, 230, 330, 430, 530 and 630 denote the double-stranded portion of the double-stranded nucleic acid molecules 100, 200, 300, 400, 500 and 600.

FIG. 2A is a graph showing GAPDH gene knockdown in MG63 cells transfected with cpRNA having different lengths of 3′-overhangs, namely, dT₂, dT₅, dT₈, as(dT₅), bl(dT₅) and (dT₅)₂, with or without CQ or MATra-si (M). The percentage of GAPDH knockdown was analyzed by RT-PCR (*P<0.005).

FIG. 2B is a graph showing GAPDH gene knockdown in MG63 cells with different cpRNA sequences with or without CQ.

FIG. 3A is a graph showing carrier-free transfection experiments using siRNA dT₂, and cpRNAs (dT₅ and dA₅) in MG63, HOB, HCT116, primary human keratinocytes and primary human fibroblast cells. The percentage of GAPDH knockdown was determined using RT-PCR experiments (***P<0.0001).

FIG. 3B illustrates a time-course plot showing uptake kinetics of Cy3-cpRNA (50 nM) as determined from flow cytometry using MG63 cells in presence or absence of 600 nM ODN2006 (pre-incubated for 1 h).

FIG. 3C illustrates fluorescence assisted cell sorting (FACS) histogram displaying uptake of Cy3-cpRNA (50 nM) in MG63, HOB, HCT116, HEK293, and MC3T3 cells after 24 h of incubation. Untreated control cells are shown in grey.

FIG. 4 is a graph showing that cellular uptake of cpRNA is an energy dependent process. HCT116, HOB and MG63 cells were transfected with dT₅ and dA₅ a 4° C. or 37° C. Cells were also transfected with negative control siRNA and cells left untreated were taken as controls and the percentage of GAPDH knockdown was analyzed by RT-PCR (***P<0.0001).

FIG. 5 is a graph illustrating cell proliferation assessed by MTS assay, 24 h post transfection. MG63 cells were transfected with siRNA sequences having different overhang lengths and negative control (NC) siRNAs at 50 nM and 100 nM, respectively, with (wM) or without MATra-si (woM). Control (C) wells were left untreated, and cell viability in control cells was defined as 1.

FIG. 6A-6C are graphs illustrating immunostimulatory effects of cpRNA. The expression level of IFN-α (FIG. 6A), IFN-β (FIG. 6B) and IFN-γ (FIG. 6C) mRNA was measured by RT-PCR at 24 h post-transfection with 50 or 100 nM of siRNA having different overhang lengths and negative control (NC), as indicated in the figures. Transfection experiments were performed in MG63 cells with (wM) or without MATra-si (woM). Expression levels were normalized to that of β-actin. The relative expression of IFN was defined as 1 in control (C) cells.

FIG. 7 (A) is a graph showing dose dependent knockdown of normal siRNA (dT₂) and siRNA (dT₅) (cpRNA). The concentration of 50 nM cpRNA displayed the highest knockdown of 80%, which did not increase with higher concentration; (B) Confocal images of MG63 cells treated with normal siRNA (dT₂) showing with localized distribution to endosome; and (C) using cpRNA (dT₅) which displayed perinuclear localization within cytosol.

FIG. 8 Durable and efficacious gene silencing induced by cpRNA (dT₅) in MG63 cells. Cells were transfected with scrambled siRNA (dT₅) (C), canonical siRNA (dT₂) with or without MATra (M), and cpRNA (dT₅) targeting GAPDH and CTNNB1 at the concentration of 50 nM. After 24, 48 and 72 h, mRNA expression of (A) GAPDH and (B) CTNNB1 was measured using RT-PCR, and the protein levels were further analysed using Western blot; β-actin was used as the loading control.

FIG. 9 illustrates GFP knockdown experiments. FIG. 9A control GFP expressing MG63 cells. FIG. 9B GFP expressing MG63 cells treated with 50 nM GFP-siRNA. FIG. 9C GFP expressing MG63 cells treated with 50 nM GFP-cpRNA.

FIG. 10 illustrates the conjugation of aldehyde modified siRNA with hydrazide modified hyaluronan or HA (A) Gel electrophoresis assay confirming the siRNA aldehyde-HA hydrazide conjugate. The first lane corresponds to normal siRNA dT₂ (siRNA); the second lane corresponds to aldehyde-modified GAPDH siRNA (dT₅) (cpRNA); the third lane shows efficient conjugation of aldehyde-modified GAPDH siRNA (dT₅) to HA by hydrazone linkage (HA-cpRNA). (B) qPCR analysis showing efficient gene knockdown upon conjugation with HA. Normal siRNA (dT₂) with transfection reagent (lipofectamine) is shown in first bar (siRNA-Lipo), followed by cpRNA (dT₅) and HA-cpRNA (dT₅) conjugates at 50 and 100 nM concentrations.

FIG. 11 is a graph showing data from transfection studies of miR-34a, miR-34a-CP and siR-34a-CP with and without transfection reagent (Lipofectamine). Real-Time PCR based functional validation of the cell-penetrating microRNA-34a, and cell-penetrating siRNA-34a in mouse preosteoblast cell line MC3T3-E1 obtained from calvarial bone (values expressed in relative fluorescence units: RU).

FIG. 12 is a graph showing data from transfection studies of miR-34a, miR-34a-CP and siR-34a-CP with and without transfection reagent having magnetic particles (MaTra). Real-Time PCR based functional validation of the cell-penetrating microRNA-34a, and cell-penetrating siRNA-34a in mouse preosteoblast cell line MC3T3-E1 obtained from calvarial bone (values expressed in relative fluorescence units: RU):

FIG. 13 is a graph that provides real-Time PCR based functional validation of miRNA-34a-CP, and siRNA-34a in MC3T3-E1 cells (values expressed in relative fluorescence units: RU) showing different levels of SOX9 expression.

FIG. 14 shows fluorescence microscopic images of MG63 cells transfected with (a) Cy-3 labeled CP-siRNA and (b) Cy-3 labeled nicked CP-siRNA. Red: Cy3-labeled siRNA; blue: DAPI-stained nuclei.

FIG. 15 is a graph that shows dose dependent cytotoxicity of 5FU and Stat3 siRNA molecules with 5FU overhang in A2780 ovarian cancer cell lines.

FIG. 16 shows the design of psiCheck2 vector expressing 3p and 5p target sequence.

FIG. 17 is a graph that shows the results of the Dual Glo assay used to analyse strand selection.

FIG. 18 is a graph that shows the results of RT-qPCR assay used to analyse strand selection.

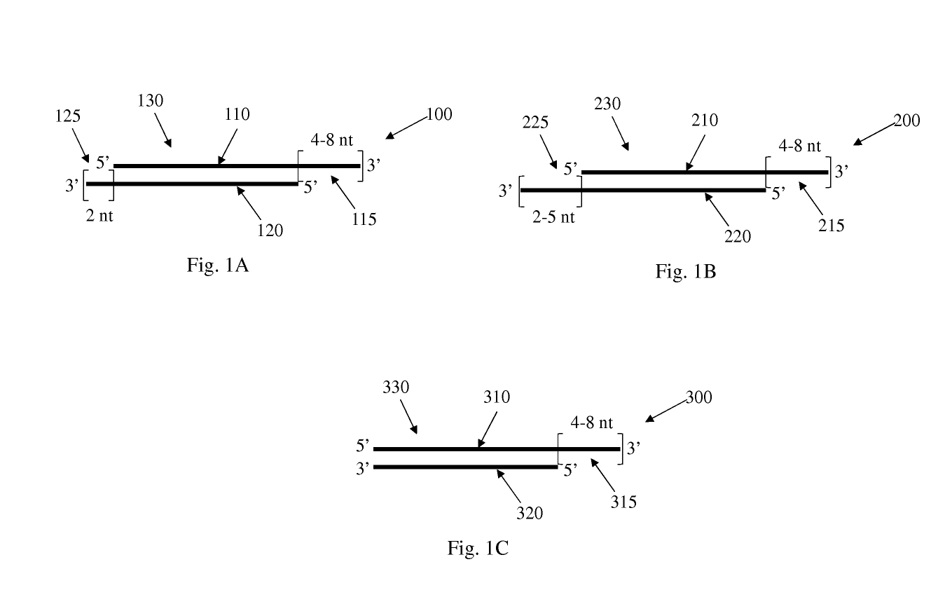

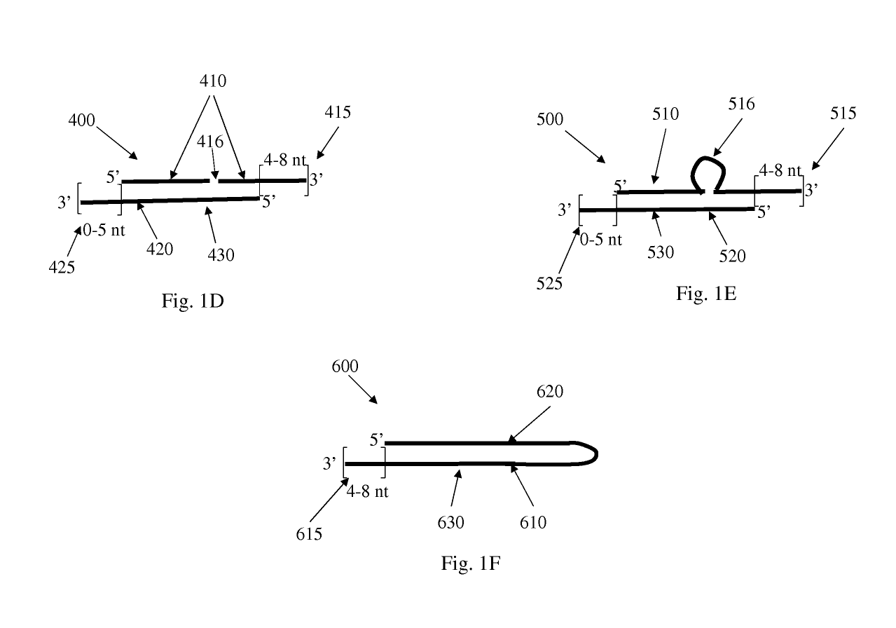

### EXAMPLES

### Example 1

### Materials and Methods

All siRNA sequences used in the present examples were high-performance liquid chromatography (HPLC) purified and purchased from Sigma-Aldrich, Sweden. The lyophilized duplexes were resuspended in RNase free water at 100 μM stock concentrations and used as it is.

The following sequences were used in the examples:

Canonical glyceraldehyde 3-phosphate dehydrogenase (GAPDH) siRNA sequences, abbreviated dT₂ herein:

(SEQ ID NO: 1)
Sense: 5′-CCG AGC CAC AUC GCU CAG A dTdT-3′
(SEQ ID NO: 2)
Antisense: 5′-UCU GAG CGA UGU GGC UCG G dTdT-3′

TABLE 3
GAPDH siRNA sequences

| | | SEQ | Abbre- |
|---|---|---|---|
| | | ID | via- |
| | | NO: | tion |
| sense 5′-3′ | CCG AGC CAC AUC GCU CAG A dT₅ | 3 | dT₅ |
| antisense | UCU GAG CGA UGU GGC UCG G dT₂ | 2 | |
| 5′-3′ | | | |
| sense 5′-3′ | CCG AGC CAC AUC GCU CAG A dT₈ | 4 | dT₈ |
| antisense | UCU GAG CGA UGU GGC UCG G dT₂ | 2 | |
| 5′-3′ | | | |
| sense 5′-3′ | CCG AGC CAC AUC GCU CAG A dT₅ | 3 | (dT₅)₂ |
| antisense | UCU GAG CGA UGU GGC UCG G dT₅ | 5 | |
| 5′-3′ | | | |
| sense 5′-3′ | CCG AGC CAC AUC GCU CAG A dA₅ | 6 | dA₅ |
| antisense | UCU GAG CGA UGU GGC UCG G dT₂ | 2 | |
| 5′-3′ | | | |
| sense 5′-3′ | CCG AGC CAC AUC GCU CAG A dG₅ | 7 | dG₅ |
| antisense | UCU GAG CGA UGU GGC UCG G dT₂ | 2 | |
| 5′-3′ | | | |
| sense 5′-3′ | CCG AGC CAC AUC GCU CAG A dC₅ | 8 | dC₅ |
| antisense | UCU GAG CGA UGU GGC UCG G dT₂ | 2 | |
| 5′-3′ | | | |
| sense 5′-3′ | CCG AGC CAC AUC GCU CAG A A₅ | 9 | rA₅ |
| antisense | UCU GAG CGA UGU GGC UCG G U₂ | 10 | |
| 5′-3′ | | | |
| sense 5′-3′ | CCG AGC CAC AUC GCU CAG A dT₂ | 1 | as |
| antisense | UCU GAG CGA UGU GGC UCG G dT₅ | 5 | (dT₅) |
| 5′-3′ | | | |
| sense 5′-3′ | CCG AGC CAC AUC GCU CAG A dT₅ | 3 | bl |
| antisense | UCU GAG CGA UGU GGC UCG G | 11 | (dT₅) |
| 5′-3′ | | | |
| sense 5′-3′ | Cy3-CCG AGC CAC AUC GCU CAG A | 12 | Cy3 |
| | dT₅ | | cpRNA |
| antisense | UCU GAG CGA UGU GGC UCG G dT₂ | 2 | |
| 5′-3′ | | | |

### GFP-siRNA Sequences:

(SEQ ID NO: 13)
Sense 5′-GCAAGCUGACCCUGAAGUUdTdT-3′
(SEQ ID NO: 14)
Antisense 5′-AACUUCAGGGUCAGCUUGCdTdT-3′

### GFP-CpRNA Sequences:

(SEQ ID NO: 15)
Sense 5′-GCAAGCUGACCCUGAAGUUdTdTdTdTdT-3′
(SEQ ID NO: 14)
Antisense 5′-AACUUCAGGGUCAGCUUGCdTdT-3′

β-Catenin siRNA Sequences

(SEQ ID NO: 16)
Sense 5′-GUA GCU GAU AUU GAU GGA C dTdT-3′
(SEQ ID NO: 17)
Antisense 5′-GUC CAU CAA UAU CAG CUA C dTdT-3′

β-Catenin cpRNA Sequences

(SEQ ID NO: 18)
Sense 5′-GUA GCU GAU AUU GAU GGA C dTdTdTdTdT-3′
(SEQ ID NO: 17)
Antisense 5′-GUC CAU CAA UAU CAG CUA C dTdT-3′

Aldehyde-Modified GAPDH cpRNA Sequences

(SEQ ID NO: 19)
Sense: 5′-CCG AGC CAC AUC GCU CAG A dTdTdTdTdTU -3′
(SEQ ID NO: 2)
Antisense: 5′-UCU GAG CGA UGU GGC UCG G dTdT-3′

Negative control siRNAs (scrambled sequence) was: Stealth RNAi™ siRNA Negative Control Lo GC (Invitrogen, part number: NC: 12935-200).

### Thermal Melting Studies

Thermal denaturation studies of siRNA was carried out in phosphate buffer, pH 7.0, containing 140 mM KCl. UV absorbance was monitored at 260 nm in the temperature range from 40 to 95° C. using a Lambda 35 UV-Vis spectrophotometer equipped with a Peltier temperature programmer with a heating rate of 0.5° C./min. Samples (1 μM RNA) were denatured at 90° C. for 3 min followed by slow cooling to 27° C. prior to the measurements. Tm values were obtained from the maxima of the first derivatives of the melting curves. All Tm values given are the averages of three independent sets of experiments (±0.3° C. error range).

### Tuning Thermodynamic Asymmetry

Canonical siRNA (19 bp+2 overhang) with the desired thermodynamic asymmetry is often identified using computational methods, which may not perfectly predict highly functional siRNA. These methods cannot be applied in certain cases, such as targeting point mutations or alternatively spliced isoforms with unique exons, because only a limited number of relevant siRNA could be obtained. Hence, siRNA sequences with the desired 5′-end thermodynamic asymmetry that would be applicable to any siRNA sequence without chemical modifications was sought. To achieve this aim, the overhang length of the 3′-end of the sense strand was extended from two nucleotides (nt) to five or eight. It was hypothesized that this strand extension (‘wagging tail’) at one end (3′-overhang in sense strand) of the siRNA duplex would induce thermodynamic destabilization, which could facilitate ATP-dependent selective unwinding and RISC recruitment of the desired strand.

The siRNA melting studies clearly revealed that increasing the overhang length using five dT repeats (dT₅) resulted in a drop in Tm of about 0.5° C. while an overhang with eight dTs (dT₈) resulted in a drop of 1.5° C. (as compared to canonical siRNA). Incorporating the dT₅ overhang at both ends (dT₅)₂ (i.e., extension of the 3′-end of the sense and antisense strands) resulted in a drop in Tm of ˜1° C. These results clearly indicate symmetrical destabilization resulting in unwinding from both ends (corresponding to a Tm drop of 0.5° C. from each end).

By changing the nt type from pyrimidines to purines, a larger drop in Tm of ˜1.5° C. was observed with five dA (dA₅) overhangs and by ˜2° C. with five dG (dG₅) overhangs (see Table 3 for details of sequences). This change is presumably the result of the more ordered single-stranded structure of dA₅ and dG₅ as compared to dT₅. Other nt modifications were also tested, and their melting characteristics are shown in Table 4.

TABLE 4
Thermal denaturation of siRNA having different 3′overhang length

| | siRNA 3′-overhang | | | | | | | | | |
|---|---|---|---|---|---|---|---|---|---|---|
| | dT₂ | dT₅ | dT₈ | (dT₅)₂ | dA₅ | dG₅ | dC₅ | rA₅ | as(dT₅) | bl(dT₅) |
| Tₘ (° C.) | 81.2 | 80.6 | 79.8 | 80.1 | 79.6 | 79.2 | 80.9 | 79.0 | 79.5 | 79.1 |
| Δ Tₘ (° C.) | | −0.6 | −1.4 | −1.1 | −1.6 | −2.0 | −0.3 | −2.2 | −1.7 | −2.1 |

Tₘ values measured as the maximum of the first derivative of the melting curve (A₂₆₀ versus temperature) recorded in medium salt buffer (7.5 mM phosphate buffer pH 7.0 containing 140 mM KCl,) with a temperature range of 40-95° C. using 1 μM concentrations of the two complementary strands. Δ Tₘ = Tₘ relative to canonical siRNA (dT₂).

### Cell Culture and Transfection

For all cell experiments, unless otherwise stated, cells were seeded at a density of 35 000 cells in a 24-well cell culture plate containing α-Minimum Essential Medium (α-MEM) (Sigma-Aldrich, Haverhill, UK) supplemented with 2 mM L-glutamine, 100 U/μL penicillin, 100 mg/μL streptomycin and 10% fetal bovine serum (heat inactivated, Sigma-Aldrich) at 37° C. with 5% CO₂ until confluence was reached.

Primary human osteoblast (HOB) cells isolated from human trabecular bone [1, 2] and keratinocytes and fibroblasts cells were isolated from skin tissue from healthy human donors. MG63 (human osteosarcoma cell line), HCT116 (human colon cancer cell line), HEK293 (human embryonic kidney 293 cells), C2C12 (mouse myoblast cell line) and MC3T3 (mouse osteoblast precursor cell line) cell lines were obtained from American Type Culture Collection, ATCC (Manassas, Va., USA). Cells were cultured until confluence was reached. The human cell experiments were approved by the local ethics committee (Ethical approval # Ups 03-561).

At 70% confluency, a day prior to transfection, the cells were seeded at a density of 35 000 cells in a 24-well cell culture plate to achieve 60-80% confluence at the day of transfection. Cell penetrating siRNAs (cpRNAs) of different lengths were transfected at 50 nM concentrations with Magnet Assisted Transfection (MATra-si) reagent for siRNAs (IBA GmbH, Göttingen, Germany) according to the manufacturers' protocols. Briefly, the siRNA was mixed with MATra-si reagent composed of supermagnetic ironoxide nanoparticles and incubated for 20 min. A complex was formed which was added to the cells to be transfected. A strong magnetic force was applied beneath the cells for 15 min. This forced the complex to be drawn into the cells by the magnetic field, and as a result the siRNA was delivered directly into the cytosol. For carrier free transfection experiments, cpRNAs were simply added to cells lines MG63, HOB, HCT116, fibroblasts and keratinocytes at 50 nM concentrations. Cells were also transfected with negative control siRNA (scrambled sequence) and cells left untreated were taken as controls. Each transfection was performed in triplicate. Post transfection, cells were incubated for 24 hours.

For the experiments involving lysosomotropic agent chloroquine (CQ), CQ was first neutralized to pH 7.4 using 1 M NaOH prior to use. MG63 cells were transfected with dT₂, dT₅, dT₈, (dT₅)₂, as(dT₅), and bl(dT₅) siRNA in presence or absence of 100 μM CQ or using MATra-si and incubated for 24 h. Similar experiments were performed for other cpRNAs namely, dA₅, dT₅, dG₅, (dT₅)₂, rA₅ and dC₅. For the control experiment, cells were either left untreated or transfected with negative control siRNA (scrambled sequence). Each transfection experiment was performed in triplicate.

### Total RNA Samples

After performing transfection experiments for 24 h, 48 h and 72 h respectively, total RNA was isolated from cells by adding 500 μl of lysis buffer (Qiagen, Germany), followed by homogenization of cell lysates using QIAshredder (Qiagen, Germany). RNA was extracted from cell lysates by RNeasy Mini Kit (Qiagen, Germany). All RNA samples were treated with DNase using TURBO-DNAfree (Ambion, USA). Agilent 2100 BioAnalyzer (Agilent Technologies, Palo Alto, Calif.) was used to confirm high quality of all RNA samples, which reports an RNA Integrity Number (RIN) that takes into account the entire electrophoretic RNA trace produced in the analysis. A RIN of greater than or equal to seven indicates the RNA is suitable for high stringency applications.

The NanoDrop ND-1000 (NanoDrop Technologies, Wilmington, Del.) was used to determine the concentrations, with resulting OD 260/280 ratios between 1.95-2.03.

### Real-Time RT-PCR Experiment

cDNA synthesis was performed in triplicate using total RNA reverse transcribed using High Capacity cDNA reverse transcription kit (Applied Biosystems, USA), with non-template control added to ensure a lack of signal in assay background. Reactions were incubated on a 96-well Applied Biosystems 9800 Fast Thermal Cycler PCR System at 37° C. for 60 min followed by 95° C. for 5 min. The real-time PCR reactions were carried out with 10 μl of 2×TaqMan® Universal PCR Master Mix, no AmpErase® UNG (Applied Biosystems, USA), 9 μl diluted cDNA, and 1 μl of TaqMan gene specific assay mix in a 20 μl final reaction volume. Reference gene beta-actin (ACTB) (Applied Biosystems, USA) was selected as control for normalization of TaqMan data. The assay included a no-template control, a standard curve of five serial dilution points (in steps of 3-fold) of cDNA mixture. Probes specific for IFN-α (Hs01022060_m1), IFN-β (Hs01077958_s1), IFN-γ (Hs00985251_m1), ACTB(Hs01060665_g1) and GAPDH (Hs02758991_g1) were purchased from Applied Biosystems. The amplification was carried out using the 7500 Fast Real-Time PCR System (Applied Biosystems, USA) using a 40-cycle program. The 7500 software automatically calculates raw Ct (cycle threshold) values. Data from samples with a Ct value equal to or below 35 were further analyzed. Samples were normalized relative to endogenous control and differences in cycle number thresholds were calculated using comparative quantitation 2^{-ΔΔcT} method (also called the ΔΔCT method), commonly used for analyzing siRNA induced gene knockdown efficiency.

The formulas used to calculate gene knockdown were as follows: First, the ΔCT was calculated as the mean cycle threshold for the target gene minus the mean cycle thresholds for the endogenous controls ACTB, each performed in triplicates: ΔCT=CT (target gene)−CT (endogenous control). Secondly, the ΔΔCT was calculated as the ΔCT of the target minus the ΔCT of negative control (NC): ΔΔCT=ΔCT (target)−ΔCT (NC). Thereafter, the percentage of knockdown of target gene was calculated as: Fold change=2^{-ΔΔcT}, then percentage of knockdown: =100×(1-fold change)

### Western Blot Analysis:

After 48 hours of transfection, cells were washed twice with ice-cold PBS and lysed in RIPA lysis buffer (50 mM Tris-HCl, pH 8.0, with 150 mM NaCl, 1.0% Igepal CA-630 (NP-40), 0.5% sodiumdeoxycholate, 0.1% SDS, and 1.0% protease inhibitor cocktail from SIGMA-ALDRICH®). Lysates were incubated on ice for 30 minutes and centrifuged at 10,000 rpm for 20 minutes to collect supernatant. Coomassie Plus—The Better Bradford Assay™ Reagent (Thermo Scientific) was used to measure protein concentrations. Thereafter, 20 μg of soluble protein was separated by SDS-PAGE and transferred to polyvinylidene difluoride membrane (Millipore). Primary antibodies against GAPDH (1:1000 dilution), β-catenin (1:1000 dilution; SIGMA-ALDRICH®), β-actin (1:1000; Cell Signaling Technology®) were used to probe the protein bands. Anti-Rabbit-HRP conjugated secondary antibodies (1:3000 dilution; R&D Systems®) were used to detect the primary antibodies, followed by the target protein visualization with EMD Millipore Immobilon™ Western Chemiluminescent HRP Substrate (ECL). Images were acquired using LI-COR Odyssey® Fc Dual-Mode Imaging system (LI-COR® Biosciences) and Image Studio Software.

### Statistical Analysis

The Student's t-test was used to determine statistical differences between pairs of groups. Two-way analysis of variance (ANOVA) was used to evaluate the statistical significance for comparisons within groups. p<0.05 (two-sided) was considered as statistically significant. Data were analyzed using GraphPad Prism software package (version 6.0).

RNAi Activity of Asymmetric siRNA

It was then explored if selective destabilization could influence RNAi activity because selective strand recruitment into RISC could improve this activity. To address this idea, a cell-based assay with human osteosarcoma cells (MG63) in which cellular components were present in physiologically relevant concentrations. As a model gene target, the housekeeping gene GAPDH (glyceraldehyde 3-phosphate dehydrogenase) was chosen, and RNAi activity was measured using quantitative RT-PCR with β-actin as the internal standard. Transfection experiments were performed with Magnet Assisted Transfection reagent (MATra-si) because this method carries reduced toxicity and does not rely on the conventional endocytosis mechanism generally observed with cationic polymer/lipid-based reagents.

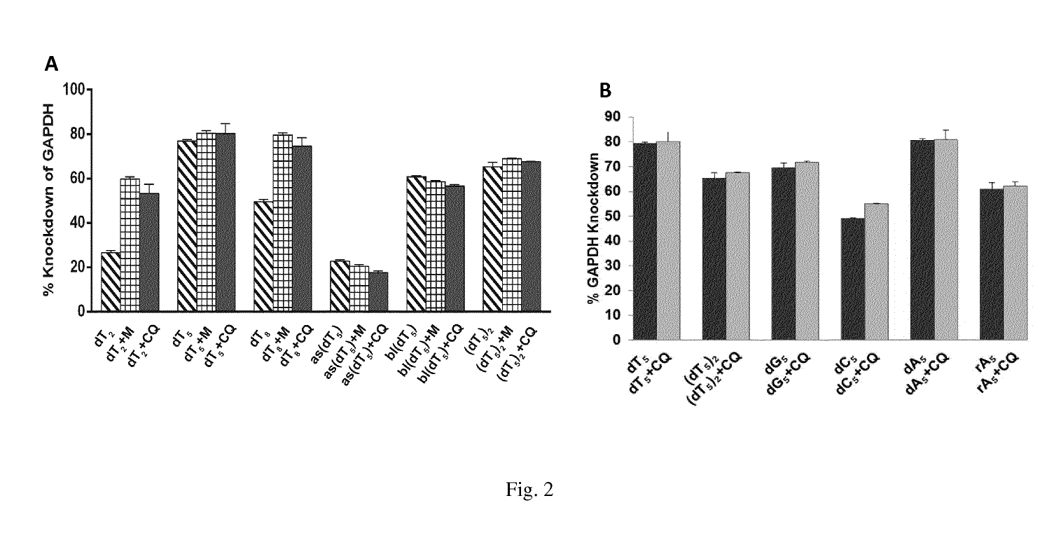

These experiments showed that an increase in overhang length to five or eight nt indeed resulted in superior gene-silencing activity (80% gene knockdown) compared to canonical siRNA (60% gene knockdown; FIG. 2A). This 20% enhancement was attributed to the thermodynamic asymmetry of cpRNA resulting in preferential recruitment of antisense strand to RISC as compared to the canonical siRNA. Interestingly, when the symmetrical siRNA (dT₅)₂ was tested, a 10% enhancement in activity (70% gene knockdown) compared to canonical siRNA was seen. Though this activity was lower than cpRNA as anticipated, the enhancement is presumably due to easier unwinding of this siRNA over canonical siRNA.

Cell-Penetrating siRNA

Carrier-free transfection experiments were then performed using siRNA with different overhang lengths (dT₂, dT₅, dT₈ and (dT₅)₂). Surprisingly, these experiments showed that the dT₅-modified siRNA sequence resulted in 80% gene knockdown (similar to MATra-si-based transfection) whereas dT₂- and dT₈-modified siRNA showed only modest 28% and 50% knockdown values, respectively (FIG. 2A). Symmetrical (dT₅)₂ modification, on the other hand, resulted in 65% gene knockdown, which was comparable to outcomes with the MATra-si-based technique (70%). This finding suggested that a siRNA design with a 5-nt overhang length (dT₅) performed all of the important steps for a siRNA-based drug, from cellular uptake to endosomal escape to selective RISC recruitment.

To further confirm the selectivity in RISC recruitment as a result of specific modification at the 3′-end, we tested GAPDH-siRNA where the dT₅ nts were incorporated at the 3′-end of antisense strand as(dT₅). This modification resulted in sharp decline in mRNA knockdown (20%) validating our hypothesis. Noteworthy, when we tested the blunt ended siRNA, bl(dT₅), where the 3′-dT₂ present at antisense strand was removed while retaining the dT₅ at the 3′-end of sense strand (Table-3), we observed a 20% decrease in activity (60% gene knockdown). These results reveal the critical parameters for RISC recruitment of siRNA.

We therefore designated siRNA with the 5-nt overhang design as cell penetrating siRNA or cpRNA. Cellular uptake of cpRNA was also confirmed by performing transfection experiments using Cy3 labeled cpRNA.

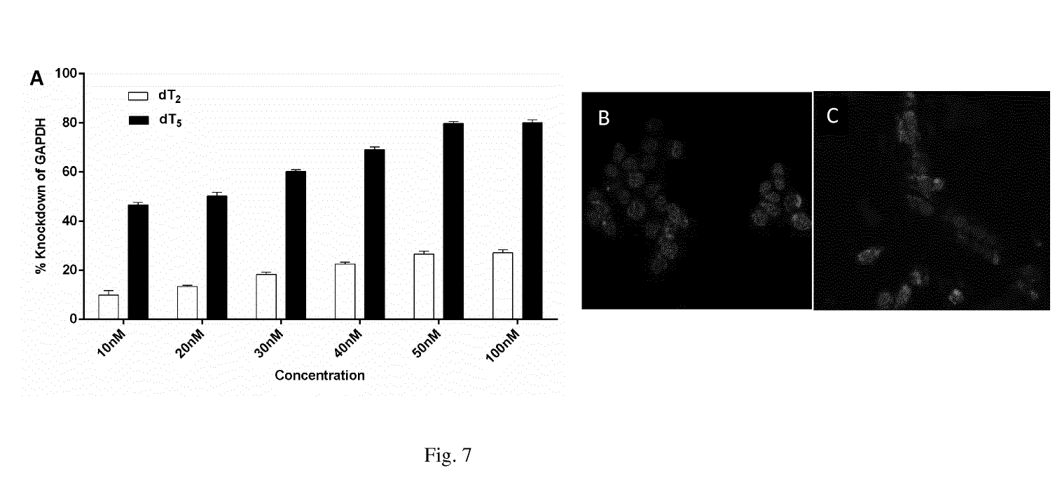

Additionally, we performed a concentration gradient study of siRNA and cpRNA (10 nM-100 nM) to determine the optimum concentration of siRNA for the transfection study. We observed highest gene knockdown (28% for canonical siRNA and 80% for dT₅ cpRNA) at the concentration of 50 nM after 24 h of incubation, which did not further increase when the concentration was increased to 100 nM (FIG. 7A). This experiment also demonstrated concentration dependent gene knockdown using cpRNA.

This intriguing result raised several important questions. (1) Is this phenomenon the result of better cellular uptake or of early release from the endosomal compartment, the sensitive region of the cell where RNA is generally trapped and degraded? (2) Is it specific to overhangs with a deoxyribose or ribose nt? (3) Is it specific to overhangs of a particular nt type (A, T, G, or C)? (4) Is it specific to a particular siRNA sequence? (5) Is it an energy-dependent process? (6) Do such modifications induce cytotoxicity or immune activation? Finally, (7) is it applicable to all cell types?

A series of experiments were performed to address these important questions. The carrier-free transfection experiment was repeated in presence of chloroquine, a lysosomotropic agent known to break the cellular endosome. To our surprise, this experiment yielded compelling evidence that cells take up all siRNA types (including canonical siRNA) but sequester them within the endosome. There, the siRNA molecules lose their activity, with the exception of siRNA bearing the 5-nt overhang (FIG. 2A).

Different purine and pyrimidine sequences in 5-nt repeats were then tested. The advantage of using such repeats is minimizing the possibility of sequence homology within the siRNA sequence (preventing secondary structures) and also limiting immune activation that is specific to dinucleotide repeats (e.g., CpG). Among different 5-nt candidates tested, siRNA with dA₅ and dT₅ overhangs demonstrated the highest RNAi activity (FIG. 2B). Of note, the ribose analogue rA₅ showed only 60% knockdown, as compared to dA₅ (80%), suggesting the presence of a specific enzymatic bias for deoxyribose over ribose nucleotides within the RISC complex. This result could also arise from differences in enzymatic degradation of single-stranded DNA versus single-stranded RNA.

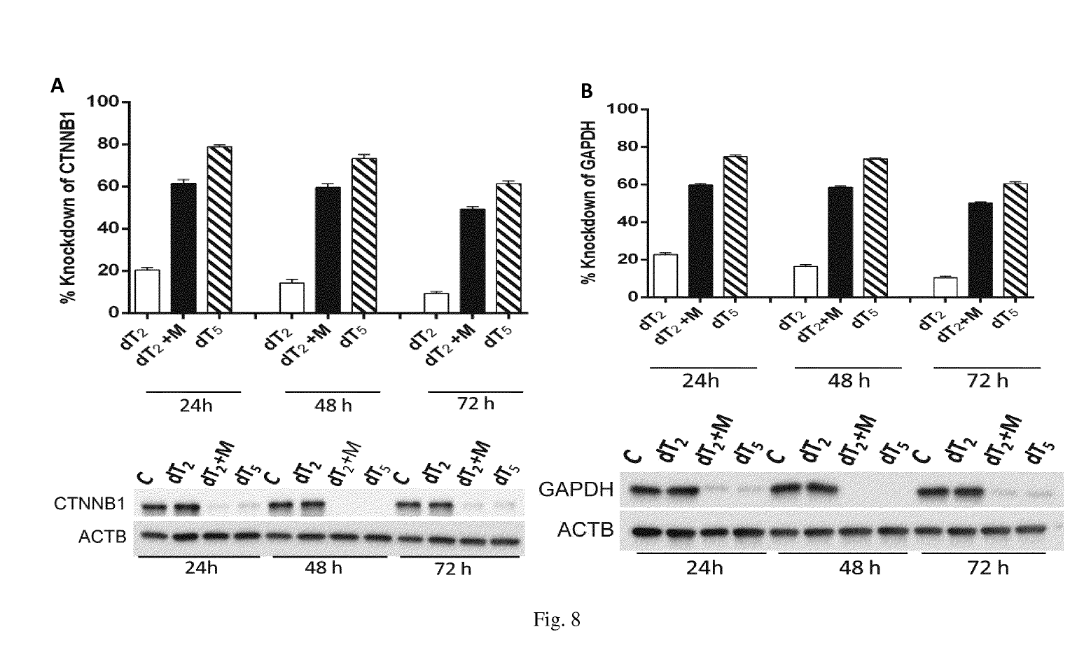

To further validate our results, we tested the gene knockdown efficiency of another gene CTNNB1 (β-catenin), which is expressed in MG63 cells. We performed a time course experiment (24, 48 and 72 h) by targeting CTNNB1 and GAPDH in MG63 cells and quantified the mRNA and protein levels using qRT-PCR and Western blot experiments respectively (FIG. 8). These experiments confirmed that cpRNA had higher gene knockdown efficiency for over 72 hrs without any transfection reagent as compared with that of canonical siRNA with transfection reagent (FIG. 8A, 8B). This result was also profound at the protein level, which showed complete knockout of CTNNB1 and GAPDH proteins at 48 h with minor expression levels at 24 and 72 h.

Versatility of cpRNA RNAi Activity

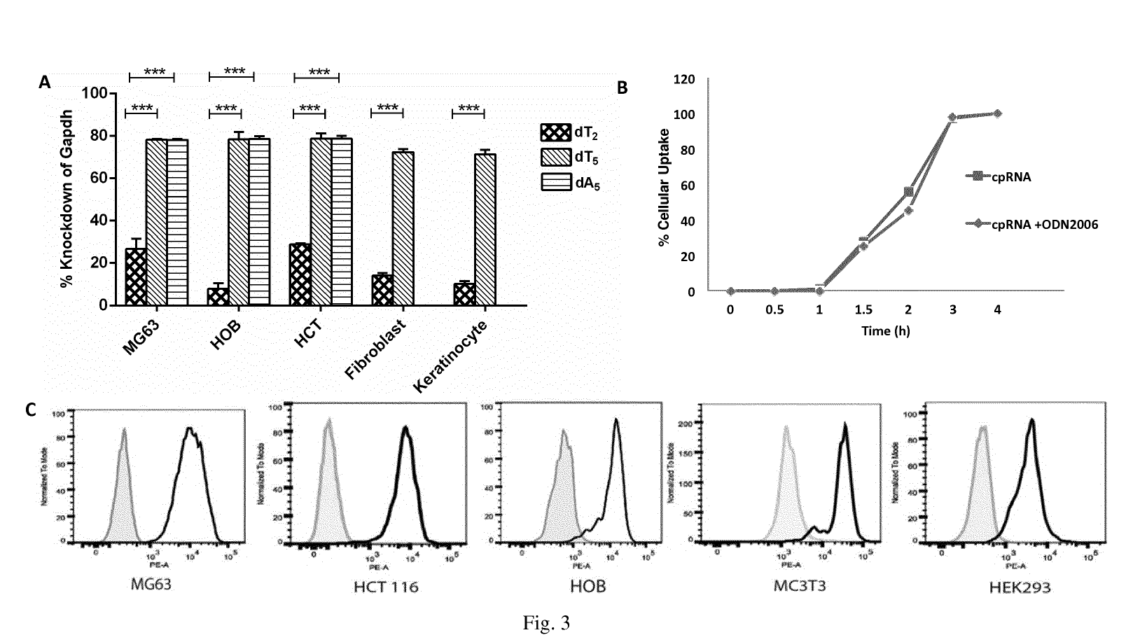

We then focused on the transfection efficiency of cpRNA in different cell types because transfection with different carriers generally works efficiently for fast-dividing cancer cells but not for hard-to-transfect primary cells. To test the transfection system with different cell types, human primary osteoblasts (HOB) were chosen as model primary cells; and human colon carcinoma cells (HCT116) and MG63 as model cancer cells. These experiments clearly demonstrated that cpRNA resulted in a 3-4-fold higher knockdown efficiency compared to canonical siRNA (FIG. 3A). This unique RNA design retained identical gene knockdown (80%) capability in different cell types, even as canonical siRNA exhibited only limited activity in primary cells (FIG. 3A).

To quantify cellular uptake in different cells, flow cytometry experiments with were performed with different cell types using Cy3-labeled cpRNA where Cy3 was incorporated at the 5′-end of the sense strand with the 3′-dT₅ overhang. These experiments confirmed near quantitative cellular uptake in almost all cells tested (FIG. 3C). This Cy3-modified cpRNA also showed 80% gene knockdown in MG63 cells, suggesting that blocking 5′-phosphorylation of the sense strand (inhibiting sense recruitment in RISC) did not further improve RNAi activity (data not shown). It also verified that hydrophobic modification at the 5′-end of the sense strand in cpRNA was well tolerated without any loss of activity.

These experiments showed that cpRNA having five nucleotide overhangs perform all necessary biochemical steps necessary for RNAi activity. These steps include, (1) cellular uptake, (2) endosomal escape, and (3) selective RISC recruitment. These results are indicated in FIGS. 2 and 3.

Cellular Uptake of cpRNA is an Energy Dependent Process

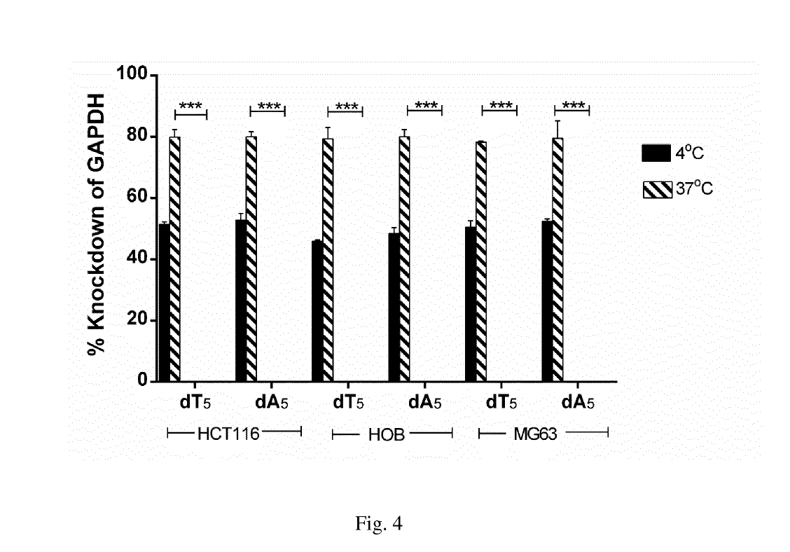

To determine, if cellular uptake of cpRNA is an energy dependent endocytosis process, transfection experiments were performed at 4° C. and 37° C. Briefly, HCT116, HOB and MG63 cells were plated and incubated with cpRNA (dT₅ or dA₅ or scrambled siRNA sequence) at 4° C. or 37° C. for 4 h. After 4 h, medium was replaced with fresh medium and the cells were incubated for additional 24 h. Untreated cells were used as controls. Each transfection was performed in triplicate. These experiments clearly showed that cellular uptake of cpRNA is an energy dependent process. Cellular uptake using these siRNA sequences was reduced by nearly 30% when experiments were performed at 4° C., see FIG. 4.

Mechanism of Cellular Penetration of cpRNA

Next, it was investigated if the cellular uptake mechanism of cpRNA occurred by endocytosis. Transfection experiments were performed with dT₅-modified cpRNA at 4° C. and found lower RNAi activity (50% gene knockdown) compared to activity at 37° C. (80%), suggesting an energy dependent endocytosis process for cellular uptake (FIG. 4). This result indicated the presence of an exquisitely sensitive, ubiquitous cell surface receptor that bound and internalized double-stranded RNA. Upon endocytosis, the 5-nt overhang length participated in an unknown endosomal escape mechanism that promoted cytosol transport of RNA. This unique mechanism was independent of nt type because every siRNA sequence with a 5-nt length showed identical efficiency compared to a chloroquine—based transfection (FIG. 2B). One possible candidate for such a receptor is the highly up-regulated Toll-like receptor (TLR)-3 that together with TLR7, TLR8, or TLR9 has been implicated in recognizing extracellular RNA and for initiating innate immune activation of viral and synthetic RNAs. TLR-7, -8 and -9, selectively bind single-stranded DNA or RNA sequences with TLR-7 and -8 preferentially bind U and G rich sequence while TLR-9 bind CpG nt repeats. TLR3, on the other hand, nonspecifically binds the 21mer RNA duplex after dimerization of the receptor to form a 2:1 TLR3-RNA complex. This receptor is ubiquitously expressed on the cell surface and within endolysosomal compartments of almost all types of mammalian cells.

Flow Cytometry Experiments Showed Efficient Cellular Uptake of cpRNA

To determine the amount of cellular uptake of cpRNA by flow cytometry, Cy3 labeled cpRNA (Table 3) was used for transfection experiments using different types of cells (MG63, HOB, HCT116, HEK293 and MC3T3). Cells were seeded at a density of 1×10⁵ cells per well and cultured for 24 hours. Thereafter, cells were transfected with 50 nM Cy3-cpRNA, with control wells left untreated. Post-transfection, cells were incubated for further 24 h at 37° C., trypsinised and washed with PBS containing 2 mM EDTA and 0.5% human serum albumin. The cells were then suspended in PBS and subjected to fluorescence analysis on a FACSCanto II (BD Biosciences) BioVis platform, scilife laboratory. The images were analyzed using FlowJo software (version 7.6; TreeStar, Ashland, Oreg.). These experiments showed that Cy3 labeled cpRNA was quantitatively taken up by all the cells, see FIG. 3C.

To determine cpRNA cellular uptake kinetics, FACS experiments were repeated as mentioned above using MG63 cells, where Cy3-cpRNA was treated for different time points. Briefly, Cy3-cpRNA (50 nM) was added to cells, followed by incubation for 0.5 h, 1 h, 1.5 h, 2 h, 3 h and 4 h respectively. The cells were subsequently trypsinised and analyzed by FACS as mentioned above. These experiments showed that cellular uptake was initiated after 1 h and finished within next 2 h (FIG. 3B).

To perform TLR3 blocking studies, seeded MG63 cells were incubated with 600 nM 3′-FITC labeled ODN2006 for 1 h, followed by addition of 50 nM cpRNA. Cells were incubated for additional 0.5 h, 1 h, 1.5 h, 2 h, 3 h and 4 h respectively, trypsinised and analyzed by FACS analysis as mentioned above. These experiments showed that cellular uptake of cpRNA is not analogus to viral RNA or other larger double stranded RNA. Addition of ODN2006 generally inhibit cellular uptake of viral RNA, which was not observed with cpRNA (FIG. 3B).

To verify if the uptake mechanism of cpRNA was analogous to viral RNA and the RNA mimic polyriboinosinic:polyribocytidylic acid or poly(I:C), blocking studies were performed using the Btype single-stranded DNA sequence ODN2006 which share a common uptake receptor. This synthetic phosphorothioate modified oligo, though initially identified as a TLR9 agonist, blocks cellular uptake of viral RNA and poly(I:C) by preferentially competing with the common RNA uptake receptor. Blocking experiment were performed by pre-incubating the cells with FITC labeled ODN2006 for 1 h, followed by the addition of Cy3-labeled cpRNA. The blocking studies and cellular uptake was monitored using FACS analysis at different time points for up to 4 h. As a control experiment, the uptake kinetics was performed in the absence of FITC labeled ODN2006. Interestingly, these experiments demonstrated that cpRNA did not follow the same mechanism that viral RNA or poly(I:C); addition of FITC-ODN2006 did not inhibit uptake of cpRNA (FIG. 3B). Furthermore, unlike poly(I:C), which exhibited fast uptake kinetics (within 5 min), cellular uptake of cpRNA was initiated after ˜1 h of incubation at 37° C., which subsequently completed in the next 2 h. These experiments verified the involvement of an alternate uptake mechanism and indicated the presence of a ubiquitous RNA capture receptor that required ˜1 h of maturation time before the endocytosis process could initiate.

Cell Proliferation Assay Indicating Absence of Cytotoxicity of cpRNA

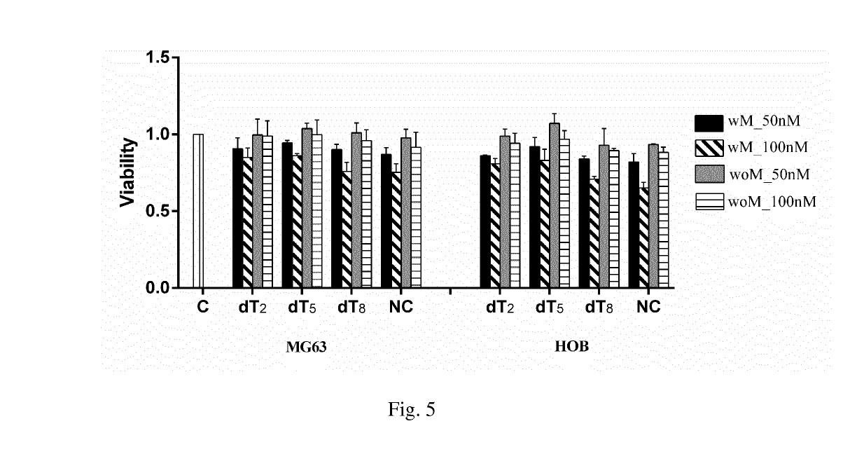

Cell proliferation was assessed by MTS (3-(4,5-dimethylthiazol-2-yl)-5-(3-carboxymethoxyphenyl)-2-(4-sulfophenyl)-2H-tetrazolium, inner salt) reagent assay: MG63 cells were seeded at a density of 35 000 cells in 24-well culture plates, and cultured for 24 hours as mentioned above. Cells were transfected with 50 nM or 100 nM concentrations of cpRNA and negative control siRNAs with or without MATra-si, while control wells were left untreated. Each transfection experiment was performed in triplicate. Post-transfection, cells were incubated for 24 hours at 37° C. The viable cells were evaluated by MTS assay, using CellTiter 96®AQueousOne Solution Cell Proliferation Assay (Promega, USA) according to the manufacturer's protocol. The enzymatic reduction in MTS to formazan was quantified by an ELISA plate reader (Thermo Scientific) at 490 nm. This experiment shows that all modified siRNA having different overhang lengths do not induce any cytotoxicity (FIG. 5).

Thus, the role of siRNA topography on immune activation and cellular toxicity was investigated because increased RNA length has been reported to cause toxicity with increased interferon (IFN)-β expression. toxicity studies were performed using the MTS assay with two concentrations of GAPDH siRNA (50 and 100 nM) with different overhang lengths (dT₂, dT₅, and dT₈) in MG63 and HOB cells and compared it with the scrambled siRNA sequence (FIG. 5). These experiments clearly showed that overhangs of different lengths do not impose toxicity on cancer cells or primary cells at high concentrations (100 nM). MATra-si-based transfection, however, resulted in slightly higher toxicity as compared to carrier-free experiments at 100 nM siRNA concentrations, which is clearly because of higher amounts of transfection agent, consistent with previous observations on carrier toxicity.

### Interferon Assay Showed No Immune Activation of Modified RNA

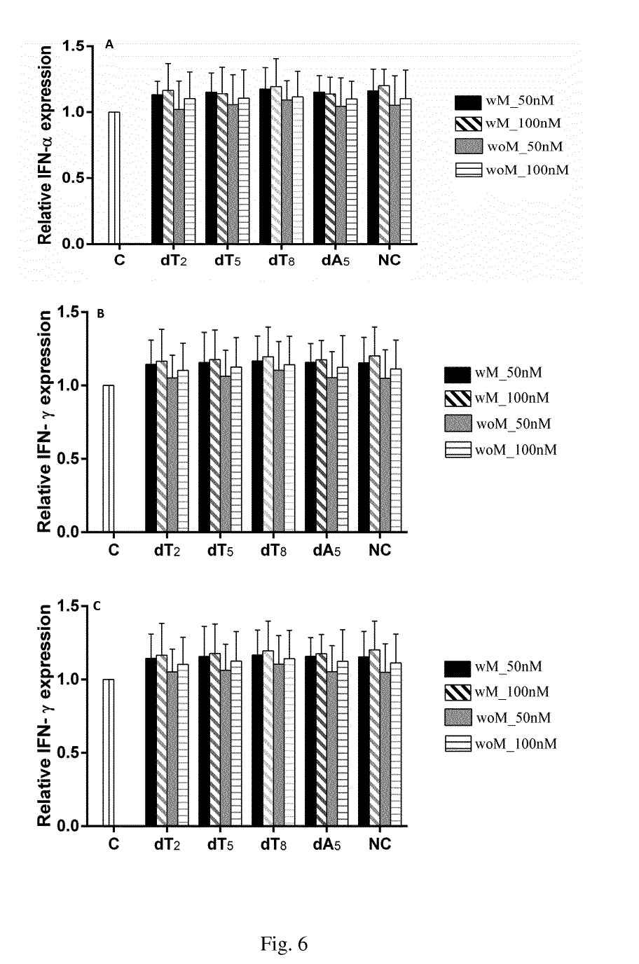

MG63 cells were transfected with 50 nM or 100 nM concentrations of cpRNA and negative control siRNAs with or without MATra-si as mentioned above. The control wells were left untreated. Each transfection experiment was performed in triplicates. After 24 h, interferon inductions were tested by RT-PCR experiments using primers specific for interferon α, β and γ. These experiments demonstrated that modified siRNAs having different overhang lengths do not trigger immune reaction (FIG. 6).

Thus, because cellular stress mainly originates from maturation of the RNA-TLR complex in the endosome or activation of RIG-1 in the cytoplasm, we investigated immune activation and IFN response using cpRNA and a scrambled sequence. The levels of Type-I (IFN-α and β) and Type-II (IFN-γ) IFN were tested via quantitative RT-PCR. IFN-α, β, and γ are activated by siRNA in different cells, in vitro and in vivo. Of note, MG63 cells are 300 to 500 fold more sensitive to IFN-β induction than other cell types such as HeLa. In these experiments, cpRNA showed no significant IFN response compared to cell-alone control or the scrambled sequence (FIG. 6). This finding is presumably due to the fast release of the cpRNA from the endolysosome compartment, preventing TLR maturation, which is a key step in initiating the TLR-mediated IFN response.

### Fluorescence Microscopy

Cellular uptake experiments were performed by plating cells (1×10⁴) in an 8 well chamber slide. At 70% confluency, the cells were transfected with 50 nM Cy3-cpRNA and incubated at 37° C. for 24 h. Control wells were left untreated. Each transfection was performed in triplicate. To evaluate the distribution of fluorescently labeled cpRNA, cells were fixed with methanol-acetone and washed with PBS. The slides were mounted using Vectashield mounting medium containing 4′, 6-diamidino-2-phenylindole (DAPI) (Vector Labs, Burlingame, Calif.) to stain the nuclei of cells. Images were captured with an Axiolmager Z1 microscope equipped with an Apotome (Carl Zeiss A B, Stockholm, Sweden).

### GFP Knockdown in MG-63 Cells

MG63 cells constitutively expressing green fluorescent protein (GFP) were seeded (1×10⁴) in an 8 well chamber slide. After 24 h the cells were transfected with 50 nM of GFP-cpRNA or GFP-siRNA as mentioned earlier. Each transfection was performed in triplicate. Control wells were left untreated. The cells were then incubated for 48 h.

To evaluate gene knockdown (GFP levels), cells were fixed with methanol-acetone and washed with PBS. Slides were mounted using Vectashield mounting medium containing DAPI to stain the nuclei in all the samples. Images were captured with an Axiolmager Z1 microscope equipped with an Apotome (Carl Zeiss A B, Stockholm, Sweden).

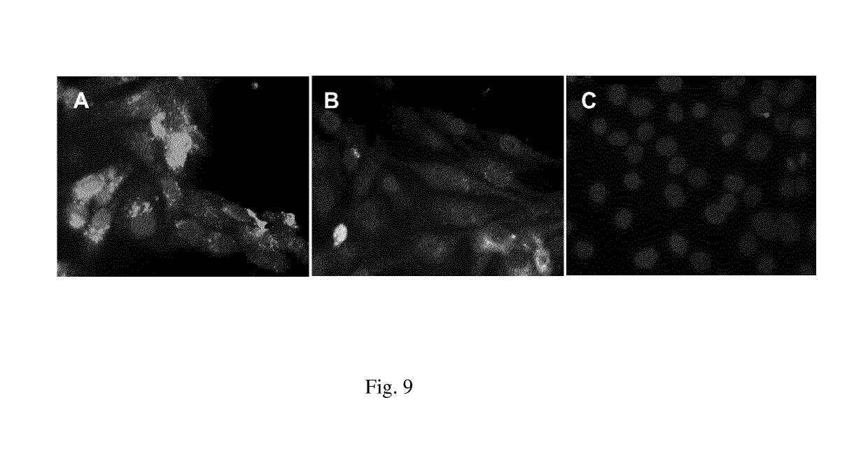

Upon incubating these GFP-expressing MG63 cells with GFP-cpRNA (50 nM), almost complete knockdown of green fluorescence was observed in 48 h (FIG. 9C). With GFP-siRNA control, on the other hand, GFP expression could be observed (FIG. 9B), exemplifying the unique property of cpRNA.

### Hyaluronan-cpRNA Conjugation and In Vitro Evaluation

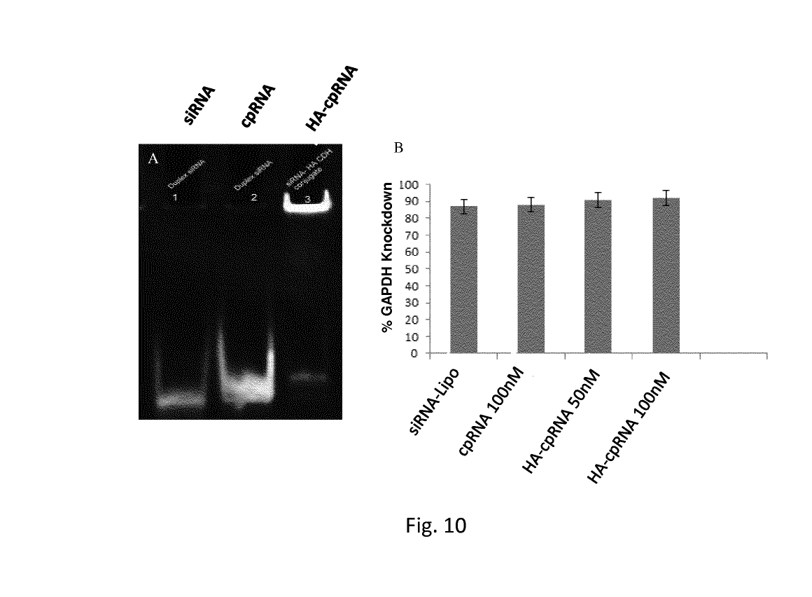

We explored HA-cpRNA conjugation method exploiting hydrazone linkages. For this purpose, we incorporated Uracil nucleoside at the 3′end of the sense strand of our modified cpRNA (see aldehyde-modified GAPDH cpRNA sequences above). The ribosugar in the terminal nucleotide was oxidized using sodium per iodate (NaIO₄) to obtain aldehyde modified cpRNA. For this purpose, 50 μl (50 nmol) of the 100 μM cpRNA was taken in a sterile eppendorf tube and 5 μl of 50 nmol sodium per iodate (NaIO₄) was added to the solution. After this cpRNA was purified using ethanol precipitation and further purified by desalting column and lyophilized. The purified cpRNA was resuspended in RNAse free water and mixed with 50 μl of hydrazide modified HA (200 nmol with 10% modification) and incubated for 1 h. The conjugation was verified by performing gel electrophoresis using 20% polyacrylamide gel (FIG. 10A).

In order to perform the functional evaluation of HA-cpRNA conjugate we selected C2C12 cell lines. The siRNA was selected to target house keeping gene GAPDH. The gene knockdown levels were evaluated using qPCR analysis.

After careful optimization, we were able to develop HA-cpRNA conjugate as could be demonstrated by gel electrophoresis assay (FIG. 10A). The conjugated product because of being high molecular weight moved slowly in the gel as compared to the unconjugated siRNA or cpRNA. Comparison of gene knockdown between different groups also showed that conjugation strategy is not detrimental for its bioactivity (FIG. 10B). The HA-cpRNA conjugate at both 50 and 100 nM concentrations gave similar gene knockdown as compared to the siRNA-Lipo control. This result is contrary to general observation of HA-siRNA conjugates which do not show gene knockdown without using cationic polymer such as PEI (Bioconjugate Chem. 2013, 24, 1201-1209).

### Example 2

### Materials and Methods

All miRNA sequences used in the present examples were high-performance liquid chromatography (HPLC) purified and purchased from Sigma-Aldrich, Sweden. The lyophilized duplexes were resuspended in RNase free water at 100 μM stock concentrations and used as it is.

miR-34a Sequences

Batch: HA07512070, sense sequence:
(SEQ ID NO: 20)
5′-CAA UCA GCA AGU AUA CUG CCC U-3′
Batch: HA07512071, antisense sequence:
(SEQ ID NO: 21)
5′-UGG CAG UGU CUU AGC UGG UUG U-3′

Cell Penetrating miR-34a (miR-34a-CP) Sequences

Batch: HA07512072, sense sequence:
(SEQ ID NO: 22)
5′-CAA UCA GCA AGU AUA CUG CCC U
dAdAdAdAdA-3′
Batch: HA07512073, antisense sequence:
(SEQ ID NO: 23)
5′-UGG CAG UGU CUU AGC UGG UUG U-3′

siR-34a-CP

Sense sequence:
(SEQ ID NO: 24)
5′-AAU CAG CUA AGU AUA CUG CCA dAdAdAdAdA-3′
Antisense sequence:
(SEQ ID NO: 25)
5′-UGG CAG UGU ACU UAG CUG GUU GU-3′

GAPDH siRNA Sequences:

CP-siRNA, sense sequence:
(SEQ ID NO: 26)
5′-CCG AGC CAC AUC GCU CAG A dTdT dTdTdT-3′
Antisense sequence:
(SEQ ID NO: 27)
5′-UCU GAG CGA UGU GGC UCG GdTdT-3′

### Nicked GAPDH CP-siRNA:

Sense sequence:
(SEQ ID NO: 28)
5′-CCG AGC CAC AUC GCU CAG A dTdT dTdTdT-3′
Nicked antisense sequence a:
(SEQ ID NO: 29)
5′-UCU GAG CGA U-3′
Nicked antisense sequence b:
(SEQ ID NO: 30)
5′-GUG GCU CGG-3′

Cell Penetrating STAT3 siRNA Sequences Having 5FU Modification (CP-siRNA-5FU5)

Sense sequence:
(SEQ ID NO: 31)
5′-GGA AGC UGC AGA AAG AUA CFF FFF-3′
(F = 5-Fluoro Uracil)
Antisense sequence:
(SEQ ID NO: 32)
5′-GUA UCU UUC UG AGC UUC CdTdT-3′

STAT3 siRNA 5FU (siRNA-5FU2)

Sense sequence:
(SEQ ID NO: 33)
5′-GGA AGC UGC AGA AAG AUA CFF-3′
(F = 5-Fluoro Uracil)
Antisense sequence:
(SEQ ID NO: 34)
5′-GUA UCU UUC UGC AGC UUC CdTdT-3′

### Cell Culture and Transfection

MC3T3-E1 cells were cultured in α-MEM (Sigma-Aldrich, Haverhill, UK) medium containing 10% FBS, 1% L-Glutamine, and 1% antibiotics (PeSt) at 37° C. with 5% CO₂. One day prior to the miRNA treatment, 35,000 cells were plated per each well of a 24-well plate. On the experiment day, cells were replaced with 2% heat inactivated-FBS containing α-MEM medium with 1% L-Glutamine, and 1% antibiotics (PeSt). miR-34a-CP (final concentration: 50 nM) was given to the each well. Mirvana scrambled miRNA mimic was used as a negative control.

### Total RNA Extraction

After 24 hours of transfection, and incubation, RNA extraction was performed by using miRCURY RNA Isolation Kit—Cell and Plant (#300100) and protocol from Exiqon, Vedbæk, Denmark. RNA samples were treated with DNase for 30 minutes at 37 degrees, thereafter inactivated with a DNase inactivation solution (TUTRBO DNase AM2238 thermofisher). Thereafter, RNA concentrations were measured using NanoDrop ND-1000 from NanoDrop Technologies, Wilmington, Del.

Quantitative RT-PCR

The cDNA was made from the total RNA using High Capacity cDNA reverse transcription kit (Applied Biosystems, USA). Reactions were performed on a 96-well Bio-Rad Thermal Cycler PCR System at 37° C. for 60 minutes, and at 95° C. for 5 min respectively. Real-time PCR reactions were performed as follows: 10 μl of 2×TaqMan® Universal PCR Master Mix, no AmpErase® UNG (Applied Biosystems, USA), 9 μl 1:5 diluted cDNA, and 1 μl of TaqMan gene specific assay mix. Final volume of the reaction mix was 20 uL. Internal control gene beta-actin (ACTB) (Applied Biosystems, USA) was selected as control for normalization of TaqMan data. The qRT-PCR reaction comprised a non-template control of cDNA in order to rule out any non-specific reading. Taqman probes for Ctnnb1, Sox9, Actb, were obtained from Applied Biosystems. The qRT-PCR amplification was carried out using the Bio-Rad qRT-PCR Thermocycler.

Structural Design of Cell-Penetrating miRNA and siRNA Design

As a typical example for carrier free transfection, we employed cell-penetrating miR-34a (miR-34a-CP) that was modified with five deoxyribonucleotides having adenosine nucleobase (dA) at the 3′-end of antisense strand. This modified version was compared with natural miR-34a and cell penetrating siRNA analog of miR-34a (siR-34a-CP). The structural design of siR-34a-CP is based on developing RNA design where the antisense strand is maintained as that of miR-34a, while the sense strand is made complementary (mismatches corrected) and five deoxyribonucleotides having adenosine nucleobase (dA) at the 3′-end of antisense strand.

Exploring Cell-Penetrating Design for Micro RNA—Functional Validation of Cell Penetrating microRNA/siRNA-34a in MC3T3E1 at Transcriptional Level

In order to achieve the transfection reagent free delivery of therapeutically potential microRNAs, we tested miR-34a and compared with miR-34a-CP. Since microRNAs unlike siRNAs have multiple targets in the cell, and there is a necessity to reduce the off-target and undesirable effects if any, we have also designed siRNA model siR-34a-CP as stated above. We have used mouse preosteoblast cell line MC3T3-E1 obtained from calvarial bone for the in-vitro functional validation of cell-penetrating molecules. On the day before transfection experiment, 35000 MC3T3-E1 cells were cultured per each well of the 24-well culture plate in alpha-Minimum Essential Medium with 10% heat inactivated FBS, 1% L-Glutamine, 1% antibiotics (Pe/St). On the experiment day, medium was removed and replaced with fresh alpha MEM medium; each well was given 50 nM of the respective microRNA/siRNA with and without transfection reagent Lipofectamine 2000, and incubated for 24 hours at 37° C. 5% CO₂. Thereafter, RNA extraction, cDNA preparation, and quantitative real-time PCR was performed by using TaqMan procedure.

Since miR-34a is known to induce osteogenic differentiation of stem cells and increase mineralization in vivo, we evaluated the differentiation of pre-osteoblast cell line MC3T3-E1 cells by quantifying the levels of β-catenin mRNA after treating with miR-34a mimic. β-Catenin is a known molecular switch that promotes osteogenic differentiation of stem cells.

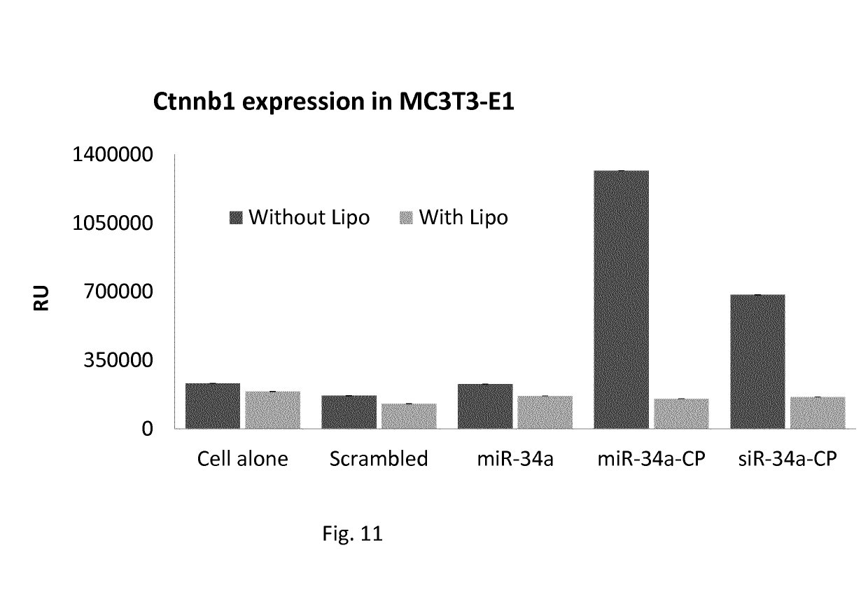

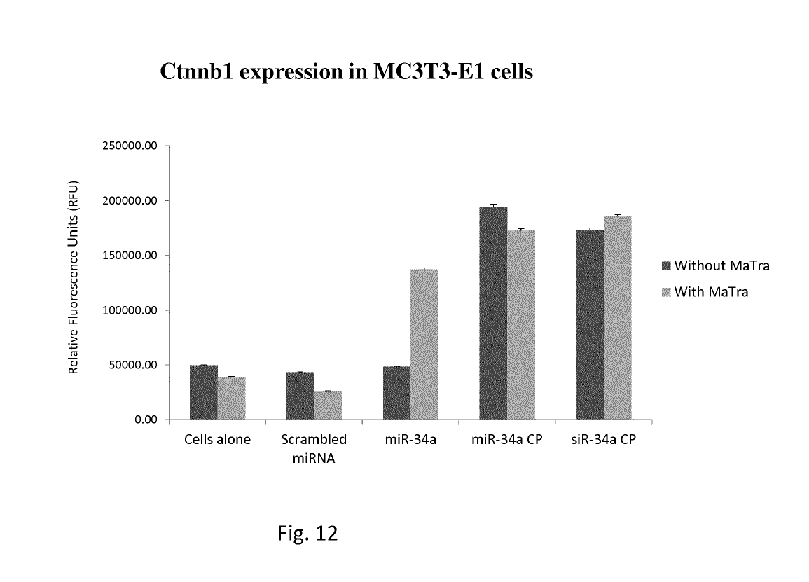

Quantitative RT-PCR results have shown the up regulation of Ctnnb1 (β-Catenin) after 24 hours of transfection in pre-osteoblast cell line MC3T3-E1 with miR-34a-CP, and siR-34a-CP alone. However, Lipofectamine 2000 based transfection of microRNA/siRNA did not show any up regulation of Ctnnb1 (FIG. 11). Since Lipofectamine 2000 did not work, we tested MaTra transfection reagent as an alternative. Quantitative RT-PCR results have shown that miR-34a induced over expression of Ctnnb1 when MC3T3-E1 cells transfected with MaTra (see FIG. 12). However miR-34a alone without transfection reagent did not show any effect on the expression levels of Ctnnb1. Gratifyingly, miR-34a-CP, and siR-34a-CP have shown up regulation of Ctnnb1 even in the absence of transfection reagent MaTra (see FIG. 12).

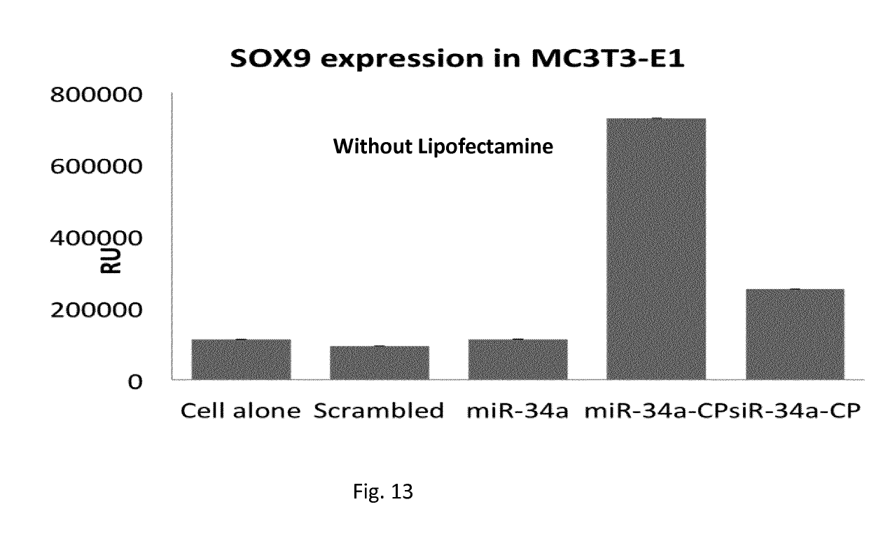

Since overexpression of Ctnnb1 is indicative of osteogenic differentiation of cells, we determined the expression of transcription factor that could be activated. For this purpose, we measured the levels of osteoblastic marker SOX9 that is implicated in endochondral ossification. The SOX9 levels were upregulated in the cell-penetrating version (miR-34a-CP) indicating an endochondral ossification mechanism as can be observed in normal osteogenesis (FIG. 13).

Imaging of Cy3-Labelled CP-siRNA and Nicked CP siRNA

MG63—human osteosarcoma cells were cultured in in α-MEM (Sigma-Aldrich, Haverhill, UK) medium containing 10% FBS, 1% L-Glutamine, and 1% antibiotics (PeSt) at 37° C. with 5% CO2. One day prior to the siRNA treatment, 35,000 cells were plated per each well of a 24-well plate. On the experiment day, cells were replaced with 10% heat inactivated-FBS containing α-MEM medium with 1% L-Glutamine, and 1% antibiotics (PeSt). Fluorescently (Cy3) labelled CP-siRNA and nicked CP-siRNA having GAPDH targeting sequence (final concentration: 50 nM) was given to the each well. After 24 hours the cells were washed with PBS and fixed with 4% formaldehyde for 20 mins at room temperature and then washed twice with PBS. DAPI (1 ug/ml in PBS) was added to cells for 5 mins and then washed with PBS. The cells were then visualized in Nikon TiU at 20×+1.5× and analysed with the Nikon Imaging software.

Gene knockdown by siRNA requires RNA to be transported to the cytosol while for correcting gene defects at the pre-mRNA levels by ‘exon skipping’, antisense DNA or RNA needs to be transported to the nucleus. The double stranded DNA or RNA is expelled out of nucleus by Exportin-5, a nuclear protein known for translocation of DNA and RNA. Thus cell-penetrating nucleic acids should be capable to deliver molecules to the nucleus without being exported out to the cytosol. Thus for this purpose, we designed a fluorescently labelled cell-penetrating double stranded RNA having a nick in the middle.

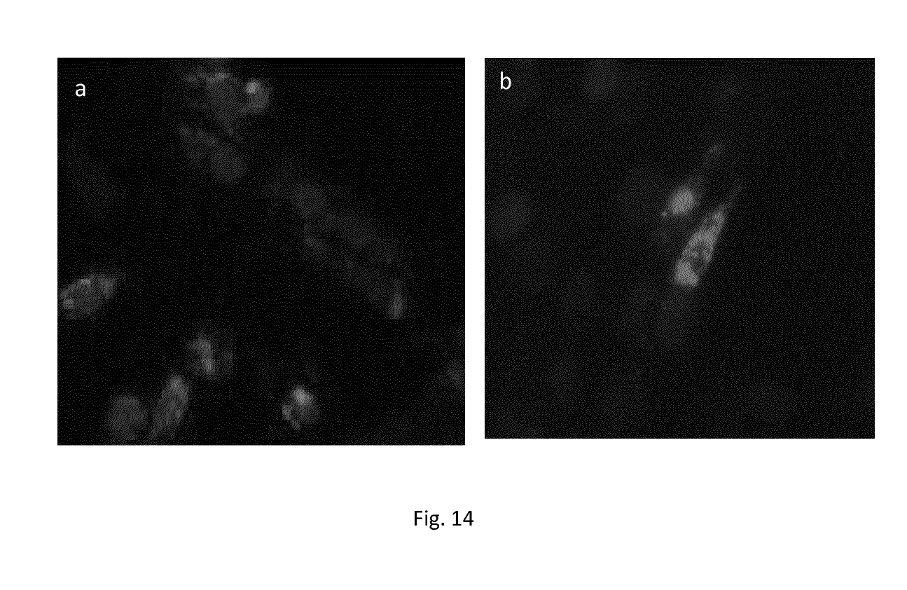

We evaluated the cellular distribution of fluorescently labelled double stranded RNA and nicked double stranded RNA by performing transfection reagent free experiments. These experiments clearly showed that CP-siRNA has perinuclear distribution as anticipated while the nicked CP-siRNA sequence has some nuclear localization (see FIG. 14). This provides a qualitative proof that nuclear delivery of RNA is indeed possible without a transfection reagent using our nicked double stranded RNA design.

Cell Penetrating STAT3 siRNA Having 5FU Modification

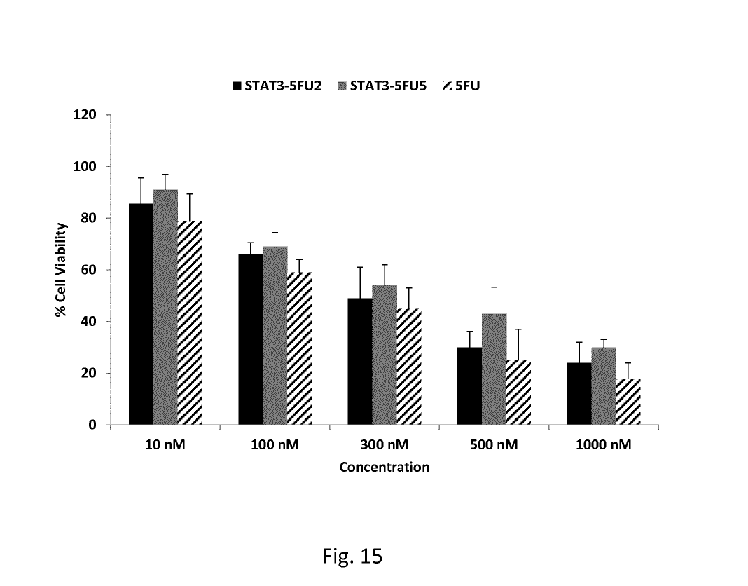

Cell viability study of 5-FU and siRNA derivatives bearing 5FU molecules were performed using, ApoTox-Glo™ Triplex Assay kit following manufacturer's protocol. Briefly, A2780 human ovarian cancer cells were seeded in 384 well BD Facon black plates (1000 cells in 50 μL/well) using automated Biomek FX pipetting workstation and incubated at 37° C. for 24 h for cell attachment. Stock solution of 5FU, siRNA-5FU2 and siRNA 5FU5 was prepared in cell culture medium (DMEM) respectively. siRNA-5FU2 and siRNA 5FU5 corresponds to Stat3 siRNA bearing 2 or 5 molecules of 5FU at the 3′end of the sense strand. After 24 h, different volume of stock solution was added to each well using non-contact acoustic dispenser (Echo 555), to obtain a gradient concentration ranging from 10 nM to 1000 nM and incubated for additional 48 h at 37° C. After 48 h, 20 μl of medium was treated with 5 μl of cell viability assay reagent, containing both GF-AFC substrate and bis-AAF-R110 substrate and incubated for 30 min at 37° C. Fluorescence values were recorded at two wavelength sets: 400Ex/505Em (Viability) and 485Ex/520Em (Cytotoxicity) using Envision Multilabel Plate Reader and the cell viability were obtained as a percentage of the untreated control (100% cell viability). As evident from FIG. 15, we observed the dose dependent cytotoxicity of 5FU and also Stat3 siRNA bearing 5FU moiety at the 3′ end of the sense strand. This clearly indicates that the bioactivity of 5FU is retained even when incorporated as an overhang in the siRNA design. All the cell experiments were measured in triplicate.

### Conclusions

The double-stranded nucleic acid molecule of the invention represents the first example of transfection agent-free cellular delivery of active siRNA, miRNA, and nicked RNA using unmodified nucleotides. Based on the presented findings, any double stranded RNA molecule, regardless of sequence, can be transformed into a self-deliverable bioactive molecule by incorporating deoxy nucleotides like A or T. These deoxy nucleotides could be replaced by biologically relevant drug molecules such as 5-fluoro uracil. This RNA design demonstrated efficient cellular uptake and endosomal escape with 3-4-fold higher bioactivity than conventional siRNA when used without any carrier molecule. This efficiency of our modified siRNA and miRNA was demonstrated in cancer cells, and hard-to-transfect primary human osteoblasts. Further findings suggested that the higher bioactivity of the siRNA and miRNA design presumably arises from preferential strand selection within the RISC complex, resulting from the selective destabilization of the 5′-end of the antisense strand. This cpRNA also induced none of the toxicity or immunogenicity associated with larger miRNA molecules. The nicked RNA design facilitated nuclear delivery of the RNA unlike normal double stranded RNA. This nuclear transport of single stranded antisense strand (obtained after in-situ disintegration of the nicked sequence) is promising for antisense or antigene therapy. Thus our invention of self-delivery of double stranded RNA sequence can be exploited for the delivery of single-stranded RNA or DNA molecule by using a nicked or mismatched double stranded structure.

### Example 3

### Materials and Methods

### Cell Culture

HCT116 was grown in Dulbecco's Modified Eagle's medium (Gibco, Invitrogen) with 10% serum and 1% PEST. Cells were cultured in incubator at 37 degree Celsius with 5% CO₂.

### Transfection

For all experiments RNAiMAX was used for transfection of both plasmid and miRNA in accordance with manufacturer's instructions, but with altered RNA and plasmid concentrations. For Dual glo assay, cells were co-transfected with 50 nM miRNA and 300 ng plasmid. For qPCR cells were transfected with 100 nM miRNA.

### Dual Glo Assay

HCT116 cells were seeded 10000 cells per well in a 96 well plate and transfected with both plasmid and miRNA 24 hours later. All transfections were performed in triplicates. The plasmids used were the dual luciferase reporter plasmid with either a 3p or 5p miR34a target-site cloned into the 3′ UTR (denoted plasmid 3 or plasmid 5). After 24 hours 75 μl of Dual glo firefly substrate (Promega) was added to each well. 100 μl of cell-lysate was moved to a microtiter plate after 10 minutes of incubation and luminescence was measured and it was followed by addition of 75 μl of Stop glo Renilla substrate (Promega) to the same plate and luminescence was measured again after 10 minutes of incubation. The Renilla luminescence values were normalized against the firefly luminescence values and expressed as a percentage of the negative control, a scrambled non-functional miRNA.

### RT-qPCR

HCT116 were seeded 50000 cells per well in 24 well plates for 24 hours and later transfected. After 24 h RNA was extracted using the mirVana miRNA isolation kit (thermofisher) in accordance with manufacturer's instructions. Total RNA concentration and purity was measured with NanoDrop 2000, and 50 ng of extracted RNA was used for cDNA synthesis with the taqman MicroRNA reverse transcription kit (thermofisher) with reverse transcriptase primers for miR34a-3p, miR34a-5p, RNU24 and U6snRNA. Expression levels of miR34a-3p and miR34a-5p were assayed through qPCR with the TaqMan® Fast Universal Master Mix (thermofisher). Values were normalized using RNU24 and U6snRNA as reference miRNAs to establish change in miRNA levels and a scrambled non-functional miRNA as a negative control. All samples were analyzed in triplicates.

### Results

Assessment of Strand Selection in miRNA34a

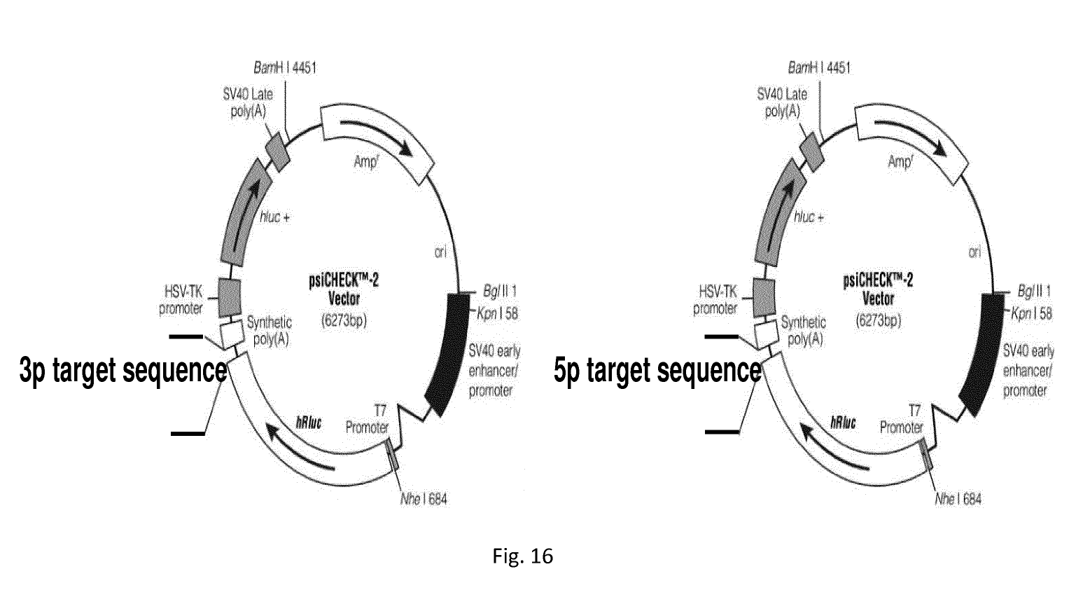

Dual glo assay and RT-qPCR were performed to identify the degree of strand selection of the modified miRNA. First, we designed two different plasmids (psiCheck2 vector) such that miRNA target-sequence (3p or 5p) is cloned into the 3′-UTR of the Renilla luciferase gene (see FIG. 16) with firefly luciferase as the internal control to normalize the Renilla luciferase expression. We estimated the levels of the individual strand recruitment (3p or 5p) using the dual-glo luminesce assay. The target-sequence corresponds exactly to either the 3p or 5p strands, and a change in luminescence can be directly attributed to the gene silencing efficiency. We also used stem-loop RT-qPCR assay to demonstrate the level of recruited 3p or 5p stands of miRNAs within the RISC complexes of a cell.

Four independent trials of dual glo assay were performed, with each of the samples in triplicates. Luminescence is shown as a percentage of the scrambled miRNA which serves as the negative control. Two different modifications were used on each of the strands of the miR34a (see Table 5). The miR34a was modified to make the 5p strand the guide strand by incorporating three deoxy thymidine residues at the 3′ end of the 3p strand (mod5a).

Since the 3p strand of the natural miR34a has a two-nucleotide 3′ overhang, the 3′ overhang length of the 3p strand of mod5a was five nucleotides. In addition, a modified molecule was created by adding five deoxy thymidine residues at the 3′ end of the 3p strand to create an overhang length of seven nucleotides (mod5b). Similarly, to make the 3p strand the guide strand, modified molecules were created by the addition three and five deoxy thymidines to the 3′ end of the 5p strand (mod3a and mod3b, respectively).

TABLE 5
miR34a sequences

| | | SEQ | |
|---|---|---|---|
| | | ID | |
| | Strand | NO: | Sequence |
| Mod3a | 3p | 20 | CAA UCA GCA AGU AUA CUG CCC U |
| | 5p | 35 | UGG CAG UGU CUU AGC UGG UUG UdTdTdT |
| Mod3b | 3p | 20 | CAA UCA GCA AGU AUA CUG CCC U |
| | 5p | 36 | UGG CAG UGU CUU AGC UGG UUG |
| | | | UdTdTdTdTdT |
| Mod5a | 3p | 37 | CAA UCA GCA AGU AUA CUG CCC UdTdTdT |
| | 5p | 21 | UGG CAG UGU CUU AGC UGG UUG U |
| Mod5b | 3p | 38 | CAA UCA GCA AGU AUA CUG CCC |
| | | | UdTdTdTdTdT |
| | 5p | 21 | UGG CAG UGU CUU AGC UGG UUG U |

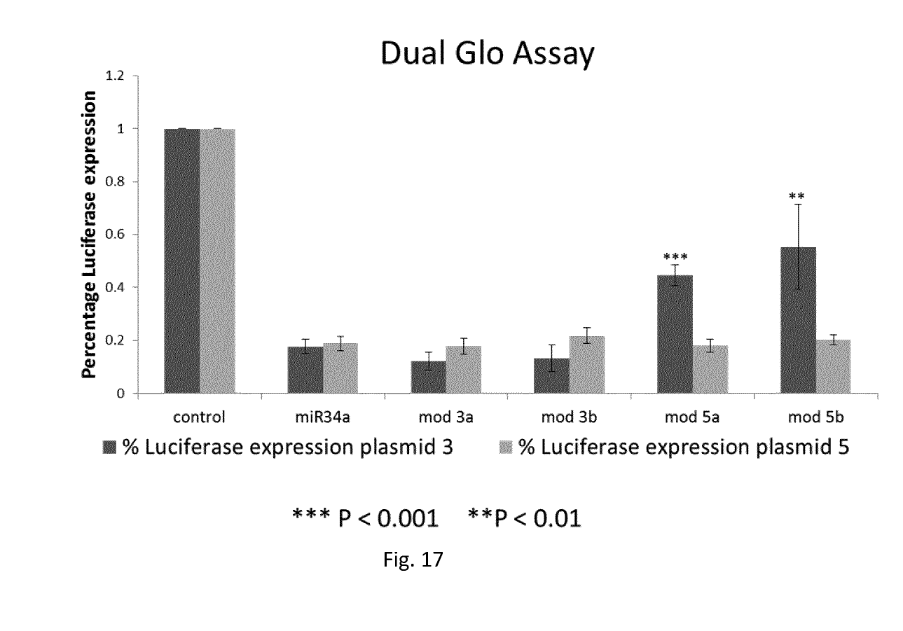

Dual glo assay showed that the mod5a and mod5b resulted in the 3p strand being less chosen as the guide strand whereas mod3a and mod3b showed a slight increase in strand selection but this was not statistically significant (FIG. 17). This data confirms that our modification does induce selective strand selection in the miR34a. To further confirm the level of each strand that was recruited, we performed stem-loop qRT-PCR experiments.

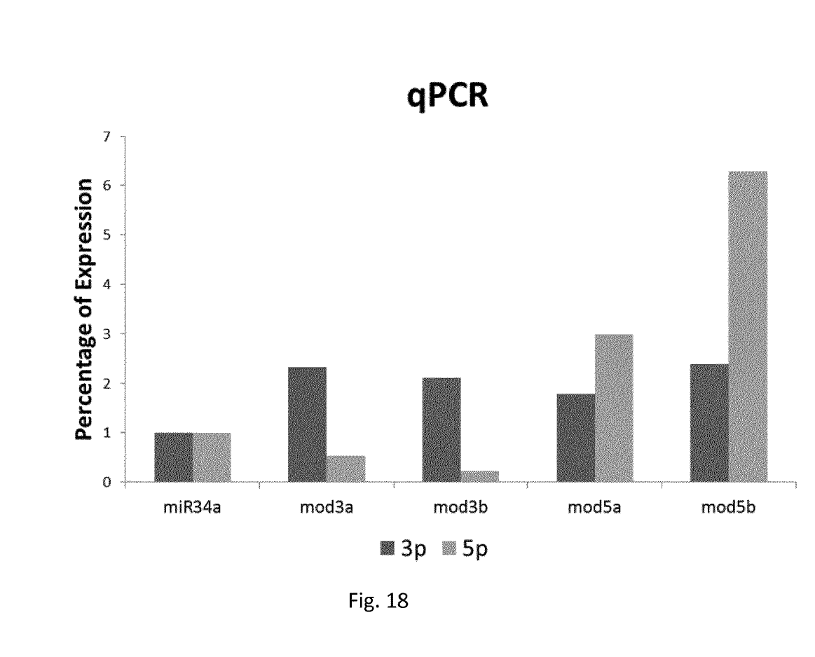

Stem-loop qRT-PCR experiments of cells transfected with modified miR34a also showed a pattern indicating strand selection (FIG. 18). The qPCR estimates the amount of the individual strands of miR34a (3p or 5p) that are recruited within the RISC complex. The strand that is not recruited is usually cleaved. These experiments also showed that all the modifications resulted in higher recruitment of the predicted strand indicating that strand selection is indeed possible using our unique design (FIG. 18).

1.-102. (canceled)

103. A double-stranded nucleic acid molecule comprising a sense strand and an antisense strand, wherein the sense strand has a 3′-overhang of from four to eight nucleotides and the antisense strand has a 3′-overhang that is shorter than the 3′-overhang of the sense strand or the antisense strand does not have a 3′-overhang, and wherein the sense strand and/or the antisense strand is a DNA strand.

104. The double-stranded nucleic acid molecule according to claim 2, wherein one or more of the nucleotides of the 3′-overhang of the sense strand and/or the 3′-overhang of the antisense strand is a therapeutic nucleotide analogue or a therapeutic nucleoside analogue.

105. A double-stranded nucleic acid molecule comprising a sense strand and an antisense strand, wherein the sense strand has a 3′-overhang of from four to eight nucleotides and the antisense strand has a 3′-overhang that is shorter than the 3′-overhang of the sense strand or the antisense strand does not have a 3′-overhang, and wherein one or more of the nucleotides of the 3′-overhang of the sense strand and/or the 3′-overhang of the antisense strand is a therapeutic nucleotide analogue or a therapeutic nucleoside analogue.

106. The double-stranded nucleic acid molecule according to claim 3, wherein the therapeutic nucleoside or nucleotide analogue is a cytotoxic nucleoside or nucleotide analogue and/or an antiviral nucleoside or nucleotide analogue.

107. The double-stranded nucleic acid molecule according to claim 3, wherein at least one of the nucleotides of the 3′-overhang of the sense strand is a therapeutic nucleotide analogue or a therapeutic nucleoside analogue.

108. The double-stranded nucleic acid molecule according to claim 1, wherein the sense strand has a 3′-overhang of from five to eight nucleotides.

109. The double-stranded nucleic acid molecule according to claim 1, wherein the antisense strand has a 3′-overhang of at least one nucleotide, optionally wherein the antisense strand has a 3′ overhang of at least two nucleotides.

110. The double-stranded nucleic acid molecule according to claim 1, wherein the sense strand is an RNA strand, optionally wherein at least one nucleotide of the 3′-overhang of the sense strand is a non-ribonucleotide.

111. The double-stranded nucleic acid molecule according to claim 1, wherein the sense strand is a DNA strand, optionally wherein at least one nucleotide of the 3′-overhang of the sense strand is a non-deoxynucleotide.

112. The double-stranded nucleic acid molecule according to claim 1, wherein the antisense strand is an RNA strand, optionally wherein at least one nucleotide in the 3′-overhang of the antisense strand is a non-ribonucleotide.

113. The double-stranded nucleic acid molecule according to claim 1, wherein the antisense strand is a DNA strand, optionally wherein at least one nucleotide of the 3′-overhang of the sense strand is a non-deoxynucleotide.

114. A double-stranded nucleic acid molecule comprising a sense strand and an antisense strand, wherein the sense strand and the antisense strand are linked together in a hairpin such that the sense strand is hybridized to the antisense strand, and wherein the double-stranded nucleic acid molecule comprises a 3′ overhang or a 5′ overhang of 4-8 nucleotides.

115. The double-stranded nucleic acid molecule according to claim 1, wherein a double-stranded portion of the double-stranded nucleic acid molecule has a length of at least 17 base pairs, at least 18 base pairs, at least 19 base pairs, or from 19 to 30 base pairs.

116. The double-stranded nucleic acid molecule according to claim 1, wherein the antisense strand is a miRNA.

117. The double-stranded nucleic acid molecule according to claim 1, wherein the antisense strand is an antisense oligonucleotide capable of gene silencing, optionally wherein the antisense oligonucleotide is a miRNA inhibitor (anti-miR), an RNAase H-dependent antisense oligonucleotide, a Piwi-interacting RNA (piRNA) or an exon-skipping antisense oligonucleotide.

118. The double-stranded nucleic acid molecule according to claim 1, wherein the double-stranded nucleic acid molecule is a double-stranded small interfering ribonucleic acid (siRNA) molecule.

119. The double-stranded nucleic acid molecule according to claim 1, wherein the antisense strand is a CRISPR guide RNA.

120. A pharmaceutical composition comprising a double-stranded nucleic acid molecule according to claim 1 and a pharmaceutically acceptable diluent.

121. The pharmaceutical composition according to claim 18, wherein either (i) the pharmaceutical composition does not comprise an agent for facilitating release of the nucleic acid molecule from the endosome into the cytosol of a cell, or (ii) the pharmaceutical composition does not comprise a cationic agent.

122. The pharmaceutical composition according to claim 19, wherein the pharmaceutical composition comprises a carrier.
